(19) United States
(12) Patent Application Publication
(10) Pub. No.: US 2023/0391823 A1
(43) Pub. Date: Dec. 7, 2023

(54) REGENERATION AND MULTIPLE USE OF DEPTH FILTERS

(71) Applicant: Hoffmann-La Roche Inc., Little Falls, NJ (US)

(72) Inventors: Roberto Falkenstein, Muenchen (DE); Christoph Feistl, Oberhausen (DE); Thorsten Lemm, Kochel am See (DE); Marc Pompiati, Penzberg (DE); Bernhard Spensberger, Eberfing (DE)

(73) Assignee: Hoffmann-La Roche Inc., Little Falls, NJ (US)

(21) Appl. No.: 18/454,131

(22) Filed: Aug. 23, 2023

Related U.S. Application Data
(63) Continuation of application No. PCT/EP2022/054210, filed on Feb. 21, 2022.

(30) Foreign Application Priority Data
Feb. 24, 2021 (EP) 21159023.7

(51) Int. Cl. C07K 1/34; B01D 37/02; B01D 39/16; B01D 39/18; B01D 39/20

(52) U.S. Cl. CPC C07K 1/34; B01D 37/025; B01D 39/163; B01D 2239/0407; B01D 39/2079; B01D 2201/085; B01D 39/18

(57) ABSTRACT

Herein is reported a method for the purification or production of a therapeutic polypeptide using the same depth filter multiple times, i.e. a depth filter which has been used before and has been regenerated.

Reported herein is a method for purifying or producing a therapeutic polypeptide, characterized in that the method comprises the following steps:
a) filtering an aqueous composition containing said therapeutic polypeptide and impurities through a depth filter, recovering the flow-through and thereby obtaining said purified therapeutic polypeptide,
b) contacting said depth filter with a regeneration solution and thereby regenerating the depth filter, and
c) repeating steps a) and b) one or more times.

The current invention is in the field of protein purification. Especially, the current invention relates to methods for purifying or producing proteins wherein the same depth filter is used multiple times. In particular, the methods of the invention involve contacting the depth filter with a regeneration solution prior to the re-use of the depth filter for removing impurities.

### BACKGROUND OF THE INVENTION

Commercial production processes for the manufacture of therapeutic biopharmaceuticals, such as monoclonal antibodies (mAbs), often utilize various clarification technologies for the removal of whole cells, cellular debris, large particulates, and colloidal matter. These clarification technologies can include centrifugation, depth filtration, chemical flocculation, and tangential or normal flow filtration methods and may be located at multiple steps within the downstream purification process. Often, the primary role of the clarification step is for the harvest of the mAb product from the cell culture broth and this may be accomplished by a combination of centrifugation followed by depth filtration. The harvest clarification step removes insoluble matter and protects the subsequent sterile filter and chromatography columns from plugging. Further downstream, depth filtration steps are also employed for secondary clarification and haze removal applications. Depth filter have also been used for achieving some level of impurity clearance, especially for the removal of process-related impurities such as host cell proteins (HCPs) and DNA, as well as product-related impurities such as aggregated mAb species (Nguyen et al, Biotechnol. J. 2019, 14, 1700771, Yigzaw et al, Biotechnol. Prog. 2006, 22, 288-296).

Generally, depth filters utilizes its depth, or thickness, for carrying out the filtration and are typically manufactured employing a material structured with a gradient density, generally having larger pores near the top and smaller pores at the bottom (when view in the direction of flow). Depth filters retain particles throughout the porous media, allowing for retention of particles both larger and smaller than the pore size. Particle retention is thought to involve both size exclusion and adsorption through hydrophobic, ionic and other interactions. Depth filters come in several different forms. A common design consists of a layer of cellulose, a porous filter aid such as diatomaceous earth (DE), and a charged polymeric resin that binds the two together. Based on those major components, depth filters remove impurities and particulate material, which is essential protection for membrane filters downstream. Several depth filtration systems are commercially available. All process-scale models e.g., Millistak+ Pod from Millipore Sigma, Stax from Pall Corporation, 3M Zeta Plus from Cuno Inc., and Sartoclear P from Sartorius Stedim Biotech can separate cells and prepare culture fluid for downstream chromatographies (Schmidt et. al, Bioprocess International, 2017).

WO 2015/031899 discloses a synthetic depth filtration media comprised of polyacrylic fibers, a precipitated silica filter aid, and a charged polymeric binder resin. This synthetic depth filter is known as the Millistak HC Pro XOSP depth filter and is commercially available as single use filter from Millipore Sigma (Bedford, MA). The XOSP depth filter has a nominal pore size rating of 0.1 microns and is intended for secondary clarification applications. Millistak+® HC Pro is a high capacity synthetic medium.

Generally, depth filters are sold for single-use. Thereby allegedly certain advantages are offered, such as, e.g., no shut down of the system is required for CIP (O'Brian et al. Bioprocess International 10, 50-67), a prerequisite in a GMP environment.

However, the filtration costs increase with single use systems compared to multi-use systems. Moreover, there is a strong need besides overall cost-reduction for ecological saving of resources.

However, the fouling mechanisms of a depth filter including pore blockage, cake formation and/or pore constriction, require an efficient regeneration protocol to get rid of the filtered impurities and to re-generate an at least equally effective depth filter.

Sodium hydroxide has become the standard for cleaning and sanitizing chromatography columns. However, some chromatography media are not compatible with sodium hydroxide. Examples of chromatography media sensitive to sodium hydroxide are: 1) chromatography media employing a protein ligand, and 2) chromatography media based on silica or glass (Application note 28-9845-64 AA GE Life Sciences). That NaOH might not be suitable for silica matrices, had also been implicated by Claesson et al., Chromatogr. A; 728 (1996) 259-270, discussing that in the case of silica based materials, NaOH treatments that render the pH value to be above pH 10 concomittantly have an intrinsic risk of hydrolyzing siloxane bonds in the silica matrix, which are the backbone of the porous structure.

In breweries, diatomaceous earth depth filter material accumulates in considerable amounts during the filtration of the wort and the stored beer. EP 0 253 233 describes a time consuming tedious regeneration protocol for the used kieselguhr employing a multistep-procedure with high temperatures and high concentrations of NaOH. Further, NaOH solutions were used in order to sterilize/sanitize Millipore depth filter prior to use US 2016/0114272. Normally, the single-use filters sold are not sanitized and need pre-flush sanitization.

Another method commonly used for the purification of depth filter is backflushing with e.g. buffer or water (e.g. in swimming pools).

### SUMMARY OF THE INVENTION

The underlying invention provides methods allowing the multiple use or re-use of a depth filter, especially silica-comprising depth filters. This is achieved by regenerating the filter material with, e.g., an acidic or alkaline solution or by pre-treating, i.e. conditioning, the depth filter prior to its first use, as well as combinations thereof. In more detail, the current invention also provides methods for increasing the efficacy of a depth filter by flushing or pre-treating it with an alkaline solution prior to its first use. By using the method according to the current invention, the efficacy of a depth filter can be increased as well as at the same time regenerated. This offers considerable economic and ecologic advantages over the single-use of depth filters.

Herein is reported a method for the purification or production of a therapeutic polypeptide using the same depth filter multiple times, i.e. wherein the same depth filter has been used at least twice and has been regenerated in between the uses. The present invention is based, at least in part, on the unexpected finding that a depth filter (such as those that contain silica as filter aid and which are intended to be a single-use, disposable depth filter) can be used multiple times, i.e. it can be regenerated, by contacting/washing/regenerating said depth filter for example with an acidic or alkaline solution according to the method of the invention. It has been found that the regenerated depth filter surprisingly maintains its ability to remove process-related impurities, such as, e.g., amongst others host cell proteins (HCPs), which are hydrolytically active, while keeping the ability to recover the main product (in comparable purity and yield as in the first use of the depth filter).

Thus, one aspect of the invention is a method for purifying a therapeutic polypeptide, characterized in that the method comprises the following steps:
a) filtering an aqueous composition containing said therapeutic polypeptide and impurities through a depth filter, recovering the flow-through and thereby purifying said therapeutic polypeptide/obtaining said purified therapeutic polypeptide,
b) contacting said depth filter (after step a)) with an acidic (regeneration) solution, thereby regenerating the depth filter, and
c) repeating steps a) and b) one or more times.

Another aspect according to the invention is a method for producing a therapeutic polypeptide, characterized in that the method comprises the following steps:
a) filtering an aqueous composition containing said therapeutic polypeptide and impurities through a depth filter, recovering the flow-through and thereby producing/obtaining said therapeutic polypeptide,
b) contacting said depth filter (after step a)) with an acidic (regeneration) solution, thereby regenerating the depth filter,
and
c) repeating steps a) and b) one or more times.

In certain embodiments of the above aspects and the other embodiments, the acidic (regeneration) solution of step b) is a solution comprising phosphoric acid.

In certain embodiments of the above aspects and the other embodiments, the acidic (regeneration) solution of step b) is a solution comprising phosphoric acid and acetic acid.

In certain embodiments of the above aspects and the other embodiments, the acidic (regeneration) solution of step b) comprises phosphoric acid in a concentration of about 0.1 M to about 0.8 M, or about 0.2 M to about 0.7 M, or about 0.4 M to about 0.6 M.

In certain embodiments of the above aspects and the other embodiments, the acidic (regeneration) solution of step b) comprises phosphoric acid in a concentration of about 0.3 M, or about 0.4 M or about 0.5 M or about 0.6 M.

In certain embodiments of the above aspects and the other embodiments, the acidic (regeneration) solution of step b) comprises acetic acid in a concentration of about 10 mM to about 2 M, or about 20 mM to about 1.5 M, or about 50 mM to about 1 M, or about 80 mM to about 800 mM.

In certain embodiments of the above aspects and the other embodiments, the acidic (regeneration) solution of step b) comprises acetic acid in a concentration of about 10 mM, or about 20 mM, or about 50 mM, or about 100 mM, or about 120 mM, or about 140 mM, or about 160 mM, or about 180 mM, or about 200 mM, or about 500 mM, or about 1 M, or about 2 M.

In certain embodiments of the above aspects and the other embodiments, the acidic (regeneration) solution of step b) comprises phosphoric acid in a concentration of about 300 mM and acetic acid in a concentration of about 167 mM.

In certain embodiments of the above aspects and the other embodiments, the acidic (regeneration) solution of step b) has a pH value of about 1 to about 5.5, or about 1 to about 5, or about 1 to about 4.5, or about 1 to about 4, or about 1 to about 3.5, or about 1 to about 3.

In certain embodiments of the above aspects and the other embodiments, the acidic (regeneration) solution of step b) has a pH value of about 1, or about 1.3, or about 1.5, or about 1.7, or about 1.9.

In certain embodiments of the above aspects and other embodiments, the acidic (regeneration) solution of step b) is an acidic aqueous buffered solution.

It has further been found that also alkaline regeneration solutions can be used in method according to the current invention.

Thus, one aspect of the invention is a method for purifying a therapeutic polypeptide, characterized in that the method comprises the following steps:
a) filtering an aqueous composition containing said therapeutic polypeptide and impurities through a depth filter, recovering the flow-through and thereby purifying said therapeutic polypeptide/obtaining said purified therapeutic polypeptide,
b) contacting said depth filter (after step a)) with an alkaline (regeneration) solution, thereby regenerating the depth filter,
and
c) repeating steps a) and b) one or more times.

One further aspect according to the invention is a method for producing a therapeutic polypeptide, characterized in that the method comprises the following steps:
a) filtering an aqueous composition containing said therapeutic polypeptide and impurities through a depth filter, recovering the flow-through and thereby producing/obtaining said therapeutic polypeptide,
b) contacting said depth filter (after step a)) with an alkaline (regeneration) solution, thereby regenerating the depth filter,
and
c) repeating steps a) and b) one or more times.

It has further been found that alkaline solutions besides being used as regeneration solutions can also be used in a pre-treatment of the depth filter (prior to its first use) resulting in a beneficial effect.

Thus, one aspect according to the invention is a method for purifying a therapeutic polypeptide, characterized in that the method comprises the following steps:
a0) contacting/incubating a depth filter with an alkaline solution,
a) filtering an aqueous composition containing said therapeutic polypeptide and impurities through said depth filter (obtained in step a0)), recovering the flow-through and thereby purifying said therapeutic polypeptide/obtaining said purified therapeutic polypeptide,
b) contacting said depth filter (after step a)) with an alkaline (regeneration) solution, thereby regenerating the depth filter,
and
c) repeating steps a) and b) one or more times.

One further aspect according to the invention is a method for producing a therapeutic polypeptide, characterized in that the method comprises the following steps:
a0) contacting/incubating a depth filter with an alkaline solution,
a) filtering an aqueous composition containing said therapeutic polypeptide and impurities through said depth filter (obtained in step a0)), recovering the flow-through and thereby obtaining said therapeutic polypeptide,
b) contacting said depth filter (after step a)) with an alkaline (regeneration) solution, thereby regenerating the depth filter,
and
c) repeating steps a) and b) one or more times.

It has further been found that aqueous buffered solutions can be used as regeneration solutions in the method according to the invention in combination with a pre-treatment of the depth filter with an alkaline solution, which results in a beneficial effect.

Thus, one aspect according to the invention is a method for purifying a therapeutic polypeptide, characterized in that the method comprises the following steps:
a0) contacting/incubating a depth filter with an alkaline solution,
a) filtering an aqueous composition containing said therapeutic polypeptide and impurities through said depth filter (obtained in step a0)), recovering the flow-through and thereby obtaining said purified therapeutic polypeptide,
b) contacting said depth filter (after step a)) with an aqueous buffered (regeneration) solution, thereby regenerating the depth filter,
and
c) repeating steps a) and b) one or more times.

One further aspect according to the invention is a method for producing a therapeutic polypeptide, characterized in that the method comprises the following steps:
a0) incubating a depth filter with an alkaline solution,
a) filtering an aqueous composition containing said therapeutic polypeptide and impurities through said depth filter (obtained in step a0)), recovering the flow-through and thereby producing/obtaining said therapeutic polypeptide,
b) contacting said depth filter (after step a)) with an aqueous buffered (regeneration) solution, thereby regenerating the depth filter,
and
c) repeating steps a) and b) one or more times.

It has further been found that water in combination with/followed by aqueous buffered solutions can be used as regeneration solutions in the method according to the invention in combination with a pre-treatment of the depth filter with an alkaline solution, which results in a beneficial effect.

Thus, one aspect according to the current invention is a method for purifying a therapeutic polypeptide, characterized in that the method comprises the following steps:
a0) contacting/incubating a depth filter with an alkaline solution,
a) filtering an aqueous composition containing said therapeutic polypeptide and impurities through said depth filter (obtained in step a0)), recovering the flow-through and thereby purifying said therapeutic polypeptide/obtaining said purified therapeutic polypeptide,
b) contacting said depth filter (after step a)) with water, followed by an aqueous buffered (regeneration) solution, thereby regenerating the depth filter,
and
c) repeating steps a) and b) one or more times.

One further aspect according to the current invention is a method for producing a therapeutic polypeptide, characterized in that the method comprises the following steps:
a0) contacting/incubating a depth filter with an alkaline solution,
a) filtering an aqueous composition containing said therapeutic polypeptide and impurities through said depth filter (obtained in step a0)), recovering the flow-through and thereby producing/obtaining said therapeutic polypeptide,
b) contacting said depth filter (after step a)) with water, followed by an aqueous buffered (regeneration) solution, thereby regenerating the depth filter,
and
c) repeating steps a) and b) one or more times.

It has further been found that water can be used as regeneration solution in combination with a pre-treatment of the depth filter with an alkaline solution, which results in a beneficial effect.

Thus, one aspect according to the current invention is a method for purifying a therapeutic polypeptide, characterized in that the method comprises the following steps:
a0) contacting/incubating a depth filter with an alkaline solution,
a) filtering an aqueous composition containing said therapeutic polypeptide and impurities through said depth filter (obtained in step a0)), recovering the flow-through and thereby purifying said therapeutic polypeptide/obtaining said purified therapeutic polypeptide,
b) contacting said depth filter (after step a)) with water, thereby regenerating the depth filter,
and
c) repeating steps a) and b) one or more times.

One further aspect according to the invention is a method for producing a therapeutic polypeptide, characterized in that the method comprises the following steps:
a0) contacting/incubating a depth filter with an alkaline solution,
a) filtering an aqueous composition containing said therapeutic polypeptide and impurities through said depth filter (obtained in step a0)), recovering the flow-through and thereby obtaining said therapeutic polypeptide,
b) contacting said depth filter (after step a)) with water, thereby regenerating the depth filter,
and
c) repeating steps a) and b) one or more times.

One further aspect according to the invention is the use of an acidic solution for regeneration of a depth filter (comprising silica) that is being used at least two times in the purification of a therapeutic polypeptide.

One further aspect according to the invention is the use of an alkaline solution for regeneration of a depth filter (comprising silica) that is being used at least two times in the purification of a therapeutic polypeptide.

In certain embodiments of the above aspects and the other embodiments, the depth filter comprises silica (as filter aid).

In certain embodiments of the above aspects and the other embodiments, the method reduces the (HCP-based/originating) enzymatic hydrolytic activity/hydrolysis activity rate.

In certain embodiments of the above aspects and the other embodiments, the method reduces/removes HCPs (host cell proteins) with enzymatic hydrolytic activity. In certain embodiments of the above aspects and the other embodiments, the method reduces the enzymatic hydrolysis activity rate in the purified therapeutic polypeptide compared to the aqueous composition (of step a) prior to the application to the filter).

In certain embodiments of the above aspects and the other embodiments, the method reduces the enzymatic hydrolysis activity rate in the purified therapeutic polypeptide by at least 25%, or by at least 30%, or by at least 35%, or by at least 40% compared to the aqueous composition (of step a) prior to the application of the filter).

In certain embodiments of the above aspects and the other embodiments, the enzymatic hydrolysis activity rate is determined by a lipase activity assay (as described herein in Example 21).

In certain embodiments of the above aspects and the other embodiments, the enzymatic hydrolysis activity rate is determined by the enzymatic hydrolysis of a substrate. In certain embodiments of the above aspects and the other embodiments, enzymatic hydrolysis activity rate is determined by monitoring the conversion of a substrate. In certain embodiments of the above aspects and the other embodiments, enzymatic hydrolysis activity rate is determined by monitoring the conversion of a fluorogenic substrate 4-Methylumbelliferyl Caprylate (4-MU-C8) by cleavage of the ester bond by hydrolases present in the sample/the purified therapeutic polypeptide into a fluorescent moiety, i.e. 4-Methylumbelliferyl (4-MU).

In certain embodiments of the above aspects and the other embodiments, the enzymatic hydrolysis activity rate is determined by the enzymatic hydrolysis of a substrate. In one embodiment, the hydrolysis rate is the hydrolysis rate of 4-Methylumbelliferyl Caprylate or a nonionic surfactant, e.g. a polysorbate, (in one embodiment polysorbate 20 or polysorbate 80).

In certain embodiments of the above aspects and the other embodiments, a pharmaceutical formulation comprising the purified/produced therapeutic polypeptide and a nonionic surfactant, e.g. a polysorbate, shows reduced hydrolysis of the nonionic surfactant, e.g. the polysorbate, compared to an identical pharmaceutical formulation comprising the aqueous composition instead of the purified/produced therapeutic polypeptide.

In certain embodiments of the above aspects and the other embodiments, the yield of the (monomeric) therapeutic polypeptide obtained in step a) using the depth filter treated according to step b) (i.e. after regeneration) is at least 80%, or at least 85%, or at least 90%, or at least 95% of the yield obtained in step a) using the depth filter for the first time, i.e. in the first filtration step.

In certain embodiments of the above aspects and the other embodiments, the yield of the (monomeric) therapeutic polypeptide obtained in step a) after performing step b) (i.e. regeneration) is at least 90% of the yield obtained in step a) using the depth filter for the first time, i.e. in the first filtration step.

In certain embodiments of the above aspects and the other embodiments, the depth filter comprises a material that is selected from the group consisting of
(i) polyacrylic fiber and silica;
(ii) cellulose fibers, diatomaceous earth, and perlite; and
(iii) cellulose fiber and charged surface groups (cationic charge modifications).

In certain embodiments of the above aspects and the other embodiments, the depth filter is selected from the group consisting of an X0SP depth filter, or a PDD1 depth filter, or aVR02 depth filter.

In certain embodiments of the above aspects and the other embodiments, the depth filter is an X0SP depth filter, or a PDD1 depth filter.

In certain embodiments of the above aspects and the other embodiments, the depth filter is contacted with the (regeneration) solution of step b) for about 20 min., for 20 min. or more, for 30 min. or more, for 40 min. or more, for 50 min. or more, or for 60 min. or more.

In certain embodiments of the above aspects and the other embodiments, the depth filter is used before a first chromatography step/prior to applying the aqueous composition to a chromatography material.

In certain embodiments of the above aspects and the other embodiments, the filter is used after the first chromatography step/after applying the aqueous composition to a chromatography material, i.e. the aqueous solution is a chromatographically purified aqueous solution.

In certain embodiments of the above aspects and the other embodiments, the therapeutic polypeptide is a recombinantly produced protein. In certain embodiments of the above aspects and the other embodiments, the therapeutic polypeptide is a recombinantly produced protein that is being formulated with a nonionic surfactant e.g. a polysorbate. In certain embodiments of the above aspects and the other embodiments, the therapeutic polypeptide is selected from the group of therapeutic polypeptides consisting of antibodies, antibody fragments, antibody fusion polypeptides, Fc-region fusion polypeptides, interferons, blood factors, cytokines, and enzymes.

In addition to the various aspects and embodiments depicted and claimed, the invention is also encompassing other embodiments having other combinations of the aspects and embodiments disclosed and claimed herein. As such, the particular features presented herein, especially presented as aspects or embodiments, can be combined with each other in other manners within the scope of the disclosed subject matter such that the disclosed subject matter includes any suitable combination of the features disclosed herein. The description of specific embodiments of the disclosed subject matter has been presented for purposes of illustration and description. It is not intended to be exhaustive or to limit the disclosed subject matter to those embodiments disclosed.

### DETAILED DESCRIPTION OF THE INVENTION

Herein is reported a method for the purification or production of a therapeutic polypeptide using the same depth filter multiple times, i.e. using a depth filter, which has been used before and has been regenerated.

The present invention is based, at least in part, on the unexpected finding that the functionality of a depth filter can be maintained after reaching its capacity limit when an acidic or alkaline solution in a method according to the invention is applied to the depth filter after its use, i.e. when the depth filter is regenerated thereby.

The regeneration of depth filters for re-use according to the current invention follows a general principle. The regeneration can be performed with regeneration solutions of different nature. In different aspects of the invention the regeneration is effected by applying
an acidic regeneration solution, or
an alkaline regeneration solution.

The acidic regeneration solution or the alkaline regeneration solution can be acidic aqueous buffered solutions or alkaline aqueous buffered solutions.

The invention is further based, at least in part, on the finding that the treatment with one of the above solutions can be effected or improved, if it is combined with an alkaline pre-treatment of the depth filter.

In the alkaline pre-treatment, the depth filter is incubated with an alkaline solution for a defined period of time prior to its first use and the regeneration is effected by one of the above regeneration solutions and in addition an aqueous buffered regeneration solution, or water, or water in combination with an aqueous buffered regeneration solution can be used.

The invention is further based, at least in part, on the finding that after alkaline pre-treatment of the depth filter regeneration of the depth filter can also be achieved by applying
an aqueous buffered regeneration solution, or
water or water in combination with an aqueous buffered regeneration solution.

The method according to the invention can be used to purify as well as to produce a therapeutic polypeptide, such as an antibody.

In more detail, it has been found that the use of regeneration solutions enable the re-use of depth filters for multiple times (cycles) for the purification of compositions comprising therapeutic polypeptides. The current inventors have shown the general applicability of the method according to the current invention with several different depth filters, different regeneration solutions as well as different antibodies/therapeutic polypeptides (in different formats) as exemplary implementation and embodiments of the invention.

FIG. 1 shows the course of the hydrolytic activity (determined with a LEAP assay according to Example 21) for multiple uses of a PDD1 depth filter (PDD1 SUPRAcap™-50 (SC050PDD1) comprising cellulose fibers, diatomaceous earth, and perlite)). An aqueous composition containing the antibody crovalimab, an anti-C5 antibody, as therapeutic polypeptide was used. In each filtration cycle (#1 to #6) the depth filter was loaded with the same amount of the antibody composition and passed through the depth filter. The resulting compositions, i.e. filtrates #1 to #6, were analyzed. In general a constant increase in the hydrolytic activity in the filtrate can be seen. In the first two cycles, the binding capacity limit of the depth filter was not yet exceeded. The second cycle (#2) represents the hydrolytic activity in the filtrate after the depth filter was re-used for the first time. No treatment of the depth filter, i.e. no regeneration, was performed in between the cycles. The hydrolytic activity increases, but only slightly, which can be expected as the binding capacity limit of the depth filter is not yet reached. In cycles #3 and onward, i.e. after the binding capacity of the depth filter has been exceeded, the depth filter is further re-used without regeneration. It can be seen that the hydrolytic activity increases significantly.

FIG. 2 shows the multiple use of a PDD1 depth filter and its ability to reduce the hydrolytic activity in an aqueous composition containing the same therapeutic polypeptide as used in the previous example as depicted in FIG. 1 (the antibody crovalimab, an anti-C5 antibody) but now with the application of different solutions in between the cycles. Also in these examples in each filtration cycle the depth filter was loaded with the same amount of the antibody composition, it was passed through the depth filter and the filtrate was analyzed. As it is the same filter type as used in the previous example (depicted in FIG. 1) with the same antibody composition and using the same load amount the binding capacity of the depth filter is reached after the second filtration cycle and exceeded in the third filtration cycle. After each cycle the depth filter is treated with a solution before the next filtration cycle.

In a first setup (see Example 11) the solution was an acidic solution (phosphoric acid; grey bars). It was surprisingly found that the depth filter can be re-used multiple times without significantly impairing its function when the depth filter was contacted with an acidic solution in between the cycles. The hydrolytic activity remains at very low levels throughout all cycles compared to the load (before depth filtration). Thus, with an acidic solution an efficient regeneration of the depth filter could be achieved.

In a second setup (see Example 10) an alkaline solution (sodium hydroxide; black dotted bars) was applied to the depth filter in between the cycles. Additionally the depth filter was per-treated before its first use by incubation with sodium hydroxide for approximately 30 minutes. Again, it was surprisingly found that the depth filter can be re-used multiple times without significantly impairing its function. The hydrolytic activity is very low compared to the load (before depth filtration) and it remains on low levels over multiple filtration cycles, i.e. the depth filter could be regenerated multiple times for re-use.

In a third setup the depth filter was treated with water and a buffered solution between the filtration cycles. No pre-treatment with an alkaline solution was done (see Example 9; grey dotted bars). After reaching the binding capacity limit a substantial increase of the hydrolytic activity in the filtrate can be observed (cf. #2 and #3 in FIG. 2).

In FIG. 3 the yield of the (monomeric) anti-C5 antibody (yield mainpeak) is depicted for the three setups shown in FIG. 2. It can be seen that for the first setup high yields can be achieved, whereby the yields remain on high levels even after multiple regeneration cycles and multiple uses. In the second setup, even an increase of the yield can be seen after multiple use. Like in the first setup, also the mainpeak yield remains on high levels. This indicates that there is no significant loss of the main, desired product when the depth filter is regenerated and re-used. Also in the third setup the yield of the mainpeak remains high.

This unexpected effect of the method according to the current invention as shown in FIGS. 1 to 3 was reproduced in an analogous manner for other, different depth filters for the same antibody. This is shown in FIGS. 4 to 6 for an X0SP depth filter (Millistak+® HC Pro X0SP comprising polyacrylic fiber and silica and in FIG. 7 to 9 for an VR02 depth filter (Zeta Plus™ Biocap VR02 comprising cellulose fiber and charged surface groups). More variations with respect to the regeneration solution, the implementation of the pre-treatment and different therapeutic proteins are presented in the examples.

For instance in example 1 a bispecific antibody is used and the depth filter is regenerated with an alkaline solution without pre-treatment. As can be seen in the Tables of Example 1, the hydrolytic activity is significantly reduced and remains on a low level. Interestingly, at first the yield decreases and it increases after four filtration cycles again, reaching higher levels than after the first filtration cycle. This drop in mainpeak yield can be circumvented by the pre-treatment of the filter with an alkaline solution (see e.g. Example 7 and Example 10 (=setup 2 in FIG. 2 above)).

A skilled person will acknowledge that the depth filter has to be equilibrated (with equilibration buffer) before the aqueous composition is applied, i.e. before it can be used. Without explicitly mentioning this, step a) includes the sub-step of contacting the depth filter with an equilibration buffer.

### SPECIFIC EMBODIMENTS OF THE INVENTION

Regeneration with an Acidic Solution

It has been found that an acidic regeneration solutions can be used in method according to the invention.

One aspect according to the invention is a method for purifying a therapeutic polypeptide, characterized in that the method comprises the following steps:
a) filtering an aqueous composition containing said therapeutic polypeptide and impurities through a depth filter, recovering the flow-through and thereby obtaining said purified therapeutic polypeptide,
b) contacting said depth filter (after step a)) with an acidic (regeneration) solution and thereby regenerating the depth filter,
and
c) repeating steps a) and b) one or more times.

One aspect according to the invention is a method for purifying a therapeutic polypeptide, characterized in that the method comprises the following steps:
a) filtering an aqueous composition containing said therapeutic polypeptide and impurities through a depth filter (comprising silica (as filter aid)), recovering the flow-through and thereby obtaining said purified therapeutic polypeptide,
b) contacting said depth filter (after step a)) with an acidic (regeneration) solution and thereby regenerating the depth filter,
and
c) repeating steps a) and b) one or more times.

A further aspect according to the invention is a method for producing a therapeutic polypeptide, characterized in that the method comprises the following steps:
a) filtering an aqueous composition containing said therapeutic polypeptide and impurities through a depth filter, recovering the flow-through and thereby obtaining said therapeutic polypeptide,
b) contacting said depth filter (after step a)) with an acidic (regeneration) solution and thereby regenerating the depth filter, and
c) repeating steps a) and b) one or more times.

In certain embodiments of the above aspects and other embodiments, the acidic regeneration solution of step b) is a solution comprising phosphoric acid.

In certain embodiments of the above aspects and other embodiments, the acidic regeneration solution of step b) is a solution comprising acetic acid.

In certain embodiments of the above aspects and other embodiments, the acidic regeneration solution of step b) is a solution comprising citric acid.

In certain embodiments of the above aspects and other embodiments, the acidic regeneration solution of step b) is a solution comprising phosphoric acid and acetic acid.

In certain embodiments of the above aspects and other embodiments, the acidic regeneration solution of step b) is a solution comprising phosphoric acid, acetic acid and benzyl alcohol.

In certain embodiments of the above aspects and other embodiments, the acidic regeneration solution of step b) comprises phosphoric acid in a concentration of about 0.1 M to about 0.8 M, or about 0.2 M to about 0.7 M, or in one preferred embodiment about 0.4 M to about 0.6 M.

In certain embodiments of the above aspects and other embodiments, the acidic regeneration solution of step b) comprises phosphoric acid in a concentration of about 0.3 M, or about 0.4 M, or about 0.5 M, or about 0.6 M.

In certain embodiments of the above aspects and other embodiments, the acidic regeneration solution comprises acetic acid in a concentration of about 10 mM to about 2 M, or about 20 mM to about 1.5 M, or about 50 mM to about 1 M, or in one preferred embodiment about 80 mM to about 800 mM.

In certain embodiments of the above aspects and other embodiments, the acidic regeneration solution of step b) comprises acetic acid in a concentration of about 10 mM, or about 20 mM, or about 50 mM, or about 100 mM, or about 120 mM, or about 140 mM, or about 160 mM, or about 180 mM, or about 200 mM, or about 500 mM, or about 1 M, or about 2 M.

In one preferred embodiment of the above aspects and other embodiments, the acidic regeneration solution of step b) comprises phosphoric acid in a concentration of about 300 mM and acetic acid in a concentration of about 167 mM.

In certain embodiments of the above aspects and other embodiments, the acidic regeneration solution of step b) is a solution comprising acetic acid in a concentration of about 50 mM to about 200 mM.

In certain embodiments of the above aspects and other embodiments, the acidic regeneration solution of step b) is a solution comprising citric acid in a concentration of about 10 mM to about 100 mM.

In certain embodiments of the above aspects and other embodiments, the acidic regeneration solution has a pH value of about 1 to about 5.5, or about 1 to about 5, or about 1 to about 4.5, or about 1 to about 4, or about 1 to about 3.5, or in one preferred embodiment about 1 to about 3.

In certain embodiments of the above aspects and other embodiments, the acidic regeneration solution of step b) has a pH value of about 1, or about 1.3, or about 1.5.

In certain embodiments of the above aspects and other embodiments, the acidic regeneration solution of step b) is an acidic aqueous buffered solution.

Regeneration Using an Alkaline Solution

It has been found that an alkaline regeneration solutions can be used in method according to the invention.

One aspect according to the invention is a method for purifying a therapeutic polypeptide, characterized in that the method comprises the following steps:
a) filtering an aqueous composition containing said therapeutic polypeptide and impurities through a depth filter, recovering the flow-through and thereby obtaining said purified therapeutic polypeptide,
b) contacting said depth filter (after step a)) with an alkaline (regeneration) solution and thereby regenerating the depth filter,
and
c) repeating steps a) and b) one or more times.

One further aspect according to the invention is a method for producing a therapeutic polypeptide, characterized in that the method comprises the following steps:
a) filtering an aqueous composition containing said therapeutic polypeptide and impurities through a depth filter, recovering the flow-through and thereby obtaining said therapeutic polypeptide,
b) contacting said depth filter (after step a)) with an alkaline (regeneration) solution and thereby regenerating the depth filter,
and
c) repeating steps a) and b) one or more times.

In certain embodiments of the above aspects and other embodiments, the alkaline regeneration solution of step b) is a solution comprising sodium hydroxide (NaOH) or potassium hydroxide (KOH).

In certain embodiments of the above aspects and other embodiments, the alkaline solution of step b) comprises NaOH in a concentration of about 0.1 M to about 1.5 M, about 0.2 M to about 1.4 M, about 0.3 M to about 1.2 M, about 0.4 M to about 1.1 M, or about 0.5 M to about 1 M.

In certain embodiments of the above aspects and other embodiments, the alkaline solution of step b) comprises NaOH in a concentration of at least about 0.01 M, at least about 0.05 M, at least about 0.1 M, at least about 0.2 M, at least about 0.3 M, at least about 0.4 M, at least about 0.5 M, at least about 0.6 M, at least about 0.7 M, at least about 0.8 M, at least about 0.9 M, at least about 1 M, at least about 1.1 M, at least about 1.2 M, at least about 1.3 M, at least about 1.4 M or at least about 1.5 M. In one preferred embodiment, the alkaline solution of step b) comprises at least about 1 M NaOH.

In certain embodiments of the above aspects and other embodiments, the alkaline regeneration solution of step b) is an alkaline aqueous buffered solution.

In certain embodiments of the above aspects and other embodiments, the alkaline solution of step b) additionally comprises sodium chloride (NaCl).

In certain embodiments of the above aspects and other embodiments, the alkaline solution of step b) comprises NaCl in a concentration of about 0.5 M to about 2.5 M, about 0.6 M to about 2.3 M, about 0.7 M to 2 M, about 0.8 M to about 1.8 M, or in one preferred embodiment about 0.9 M to about 1.5 M.

In certain embodiments of the above aspects and other embodiments, the alkaline solution of step b) comprises NaCl in a concentration of about 0.5 M, about 0.7 M, about 0.8 M, or in one preferred embodiment about 1 M.

In certain embodiments of the above aspects and other embodiments, the alkaline (regeneration) solution of step b) has a pH value of about 9 to 14, about 9.5 to 14, or in one preferred embodiment about 10 to 14.

In certain embodiments of the above aspects and other embodiments, the alkaline regeneration solution of step b) has a pH value of about 9 or more, about 9.5 or more, in one preferred embodiment about 10 or more, about 10.5 or more, or about 11 or more.

In certain embodiments of the above aspects and other embodiments, the method further includes prior to step a) the step a0) of incubating a depth filter comprising silica (as filter aid) with an alkaline solution.

The skilled person understands that the pre-incubation/pre-treatment time (contact time) with the alkaline solution can vary depending on the concentration and/or the pH value and/or the flow of the alkaline solution.

In certain embodiments of the above aspects and other embodiments, the pre-incubation is with an NaOH solution of 100 mM to 1.2 M. In certain embodiments of the above aspects and other embodiments, the pre-incubation is with an NaOH solution for 30 minutes to 4.5 hours. In one embodiment of the above aspects and other embodiments, the pre-incubation is with an at least 100 mM NaOH alkaline solution for at least about 30 minutes. In one preferred embodiment of the above aspects and other embodiments, the pre-incubation is with a 1 M NaOH alkaline solution for about 4 hours.

In certain embodiments of the above aspects and other embodiments, the pre-incubation is with at least 50 L, or 50 L to 200 L, or 100 L to 150 L of the alkaline solution per m² of depth filter area. In one preferred embodiment of the above aspects and other embodiments, the pre-incubation is with 100 L of the alkaline solution per m² of depth filter area (100 L/m²).

Regeneration with an Alkaline Solution and Alkaline Pre-Treatment

It has been found that alkaline solutions can be used as regeneration solutions and that a pre-treatment of the depth filter with an alkaline solution has a beneficial effect.

One aspect according to the invention is a method for purifying a therapeutic polypeptide, characterized in that the method comprises the following steps:
a0) incubating a depth filter with an alkaline solution,
a) filtering an aqueous composition containing said therapeutic polypeptide and impurities through said depth filter (obtained in step a0)), recovering the flow-through and thereby obtaining said purified therapeutic polypeptide,
b) contacting said depth filter (after step a)) with an alkaline (regeneration) solution and thereby regenerating the depth filter, and
c) repeating steps a) and b) one or more times.

One aspect according to the invention is a method for purifying a therapeutic polypeptide, characterized in that the method comprises the following steps:
a0) incubating a depth filter (comprising silica (as filter aid)) with an alkaline solution,
a) filtering an aqueous composition containing said therapeutic polypeptide and impurities through said depth filter (obtained in step a0)), recovering the flow-through and thereby obtaining said purified therapeutic polypeptide,
b) contacting said depth filter (after step a)) with an alkaline (regeneration) solution and thereby regenerating the depth filter,
and
c) repeating steps a) and b) one or more times.

One further aspect according to the invention is a method for producing a therapeutic polypeptide, characterized in that the method comprises the following steps:
a0) incubating a depth filter with an alkaline solution,
a) filtering an aqueous composition containing said therapeutic polypeptide and impurities through said depth filter (obtained in step a0)), recovering the flow-through and thereby obtaining said therapeutic polypeptide,
b) contacting said depth filter (after step a)) with an alkaline (regeneration) solution and thereby regenerating the depth filter,
and
c) repeating steps a) and b) one or more times.

In certain embodiments of the above aspects and other embodiments, the alkaline solution of step a0) is a solution comprising NaOH or KOH.

In certain embodiments of the above aspects and other embodiments, the alkaline regeneration solution of step b) is a solution comprising NaOH or KOH.

In certain embodiments of the above aspects and other embodiments, the alkaline solution of step a0) or step b) comprises NaOH in a concentration of about 0.1 M to about 1.5 M, about 0.2 M to about 1.4 M, about 0.3 M to about 1.2 M, about 0.4 M to about 1.1 M, about 0.5 M to about 1 M. In one preferred embodiment, the alkaline solution of step a0) and b) comprises NaOH in a concentration of about 0.9 M to about 1.1 M.

In certain embodiments of the above aspects and other embodiments, the alkaline solution of step a0) or step b) comprises NaOH in a concentration of at least about 0.05 M, at least about 0.1 M, at least about 0.2 M, at least about 0.3 M, at least about 0.4 M, at least about 0.5 M, at least about 0.6 M, at least about 0.7 M, at least about 0.8 M, in one preferred embodiment at least about 0.9 M, at least about 1 M, at least about 1.1 M, at least about 1.2 M, at least about 1.3 M, at least about 1.4 M, or at least about 1.5 M.

In certain embodiments of the above aspects and other embodiments, the pre-incubation is with an NaOH solution of 100 mM to 1.2 M. In certain embodiments of the above aspects and other embodiments, the pre-incubation is with an NaOH solution for 30 minutes to 4.5 hours. In one embodiment of the above aspects and other embodiments, the pre-incubation is with an at least 100 mM NaOH alkaline solution for at least about 30 minutes. In one preferred embodiment of the above aspects and other embodiments, the pre-incubation is with a 1 M NaOH alkaline solution for about 4 hours.

In certain embodiments of the above aspects and other embodiments, the pre-incubation is with at least 50 L, or 50 L to 200 L, or 100 L to 150 L of the alkaline solution per m² of depth filter area. In one preferred embodiment of the above aspects and other embodiments, the pre-incubation is with 100 L of the alkaline solution per m² of depth filter area (100 L/m²).

In certain embodiments of the above aspects and other embodiments, the alkaline regeneration solution of step b) is an alkaline aqueous buffered solution.

In certain embodiments of the above aspects and other embodiments, the alkaline solution of step a0) and/or step b) additionally comprises NaCl.

In certain embodiments of the above aspects and other embodiments, the alkaline solution of step a0) or step b) comprises NaCl in a concentration of about 0.5 M to about 2.5 M, about 0.6 M to about 2.3 M, about 0.7 M to about 2 M, about 0.8 M to about 1.8 M, or in one preferred embodiment about 0.9 M to about 1.5 M.

In certain embodiments of the above aspects and other embodiments, the alkaline solution of step a0) or step b) comprises NaCl in a concentration of about 0.5 M, about 0.7 M, about 0.8 M, or in one preferred embodiment about 1 M.

Regeneration with an Aqueous Buffered Solution and Alkaline Pre-Treatment

It has been found that aqueous buffered solutions can be used as regeneration solutions in the method according to the invention and that a pre-treatment of the depth filter with an alkaline solution has a beneficial effect.

One aspect according to the invention is a method for purifying a therapeutic polypeptide, characterized in that the method comprises the following steps:
a0) incubating a depth filter with an alkaline solution,
a) filtering an aqueous composition containing said therapeutic polypeptide and impurities through said depth filter (obtained in step a0)), recovering the flow-through and thereby obtaining said purified therapeutic polypeptide,
b) contacting said depth filter (after step a)) with an aqueous buffered (regeneration) solution and thereby regenerating the depth filter,
and
c) repeating steps a) and b) one or more times.

One further aspect as reported herein is a method for producing a therapeutic polypeptide, characterized in that the method comprises the following steps:
a0) incubating a depth filter with an alkaline solution,
a) filtering an aqueous composition containing said therapeutic polypeptide and impurities through said depth filter (obtained in step a0)), recovering the flow-through and thereby obtaining said therapeutic polypeptide,
b) contacting said depth filter (after step a)) with an aqueous buffered (regeneration) solution and thereby regenerating the depth filter, and
c) repeating steps a) and b) one or more times.

In certain embodiments of the above aspects and other embodiments, the aqueous buffered regeneration solution of step b) is the equilibration buffer/the buffer used for equilibrating the depth filter.

In certain embodiments of the above aspects and other embodiments, the equilibration buffer has a pH value of about 3.5 to about 8, or about 3.5 to about 6, or in a preferred embodiment about 4 to about 5.5. In one preferred embodiment, the equilibration buffer has a pH value of pH 4+/−0.2. In one preferred embodiment, the equilibration buffer has a pH value of pH 5.5+/−0.2.

In certain embodiments of the above aspects and other embodiments, the equilibration buffer comprises 150 mM acetic acid/tris (hydroxymethyl) amino methane.

In certain embodiments of the above aspects and other embodiments, the alkaline solution of step a0) is a solution comprising NaOH or KOH.

In certain embodiments of the above aspects and other embodiments, the alkaline solution of step a0) comprises NaOH in a concentration of about 0.1 M to about 1.5 M, about 0.2 M to about 1.4 M, about 0.3 M to about 1.2 M, about 0.4 M to about 1.1 M, about 0.5 M to about 1 M. In one preferred embodiment, the alkaline solution of step a0) comprises NaOH in a concentration of about 0.9 M to about 1.1 M.

In certain embodiments of the above aspects and other embodiments, the alkaline solution of step a0) comprises NaOH in a concentration of at least about 0.05 M, at least about 0.1 M, at least about 0.2 M, at least about 0.3 M, at least about 0.4 M, at least about 0.5 M, at least about 0.6 M, at least about 0.7 M, at least about 0.8 M, in one preferred embodiment at least about 0.9 M, at least about 1 M, at least about 1.1 M, at least about 1.2 M, at least about 1.3 M, at least about 1.4 M, or at least about 1.5 M.

In certain embodiments of the above aspects and other embodiments, the pre-incubation is with an NaOH solution of 100 mM to 1.2 M. In certain embodiments of the above aspects and other embodiments, the pre-incubation is with an NaOH solution for 30 minutes to 4.5 hours. In one embodiment of the above aspects and other embodiments, the pre-incubation is with an at least 100 mM NaOH alkaline solution for at least about 30 minutes. In one preferred embodiment of the above aspects and other embodiments, the pre-incubation is with a 1 M NaOH alkaline solution for about 4 hours.

In certain embodiments of the above aspects and other embodiments, the pre-incubation is with at least 50 L, or 50 L to 200 L, or 100 L to 150 L of the alkaline solution per m² of depth filter area. In one preferred embodiment of the above aspects and other embodiments, the pre-incubation is with 100 L of the alkaline solution per m² of depth filter area (100 L/m²).

In certain embodiments of the above aspects and other embodiments, the alkaline regeneration solution of step b) is an alkaline aqueous buffered solution.

In certain embodiments of the above aspects and other embodiments, the alkaline solution of step a0) additionally comprises NaCl.

In certain embodiments of the above aspects and other embodiments, the alkaline solution of step a0) comprises NaCl in a concentration of about 0.5 M to about 2.5 M, about 0.6 M to about 2.3 M, about 0.7 M to about 2 M, about 0.8 M to about 1.8 M, or about 0.9 M to about 1.5 M. In one preferred embodiment, the alkaline solution of step a0) comprises NaOH in a concentration of about 0.9 M to about 1.1 M.

In certain embodiments of the above aspects and other embodiments, the alkaline solution of step a0) comprises NaCl in a concentration of at least about 0.5 M, at least about 0.7 M, at least about 0.8 M, at least about 0.9 M, or in one preferred embodiment at least about 1 M.

In certain embodiments of the above aspects and other embodiments, the alkaline regeneration solution of step a0) has a pH value of about 9 to 14, about 9.5 to 14, or about 10 to 14.

In certain embodiments of the above aspects and other embodiments, the alkaline regeneration solution of step a0) has a pH value of about 9 or more, about 9.5 or more, in one preferred embodiment about 10 or more, about 10.5 or more, or about 11 or more.

Regeneration with Water Followed by an Aqueous Buffered Solution and Alkaline Pre-Treatment

It has been found that water in combination with/followed by aqueous buffered solutions can be used as regeneration solutions in the method according to the invention and that a pretreatment of the depth filter with an alkaline solution has a beneficial effect.

One aspect as reported herein is a method for purifying a therapeutic polypeptide, characterized in that the method comprises the following steps:
a0) incubating a depth filter with an alkaline solution,
a) filtering an aqueous composition containing said therapeutic polypeptide and impurities through said depth filter (obtained in step a0)), recovering the flow-through and thereby obtaining said purified therapeutic polypeptide,
b) contacting said depth filter (after step a)) with water, followed by an aqueous buffered (regeneration) solution and thereby regenerating the depth filter, and
c) repeating steps a) and b) one or more times.

One further aspect as reported herein is a method for producing a therapeutic polypeptide, characterized in that the method comprises the following steps:
a0) incubating a depth filter with an alkaline solution,
a) filtering an aqueous composition containing said therapeutic polypeptide and impurities through said depth filter (obtained in step a0)), recovering the flow-through and thereby obtaining said therapeutic polypeptide,
b) contacting said depth filter (after step a)) with water, followed by an aqueous buffered (regeneration) solution and thereby regenerating the depth filter, and
c) repeating steps a) and b) one or more times.

In certain embodiments of the above aspects and other embodiments, the alkaline solution of step a0) is a solution comprising NaOH or KOH.

In certain embodiments of the above aspects and other embodiments, the alkaline solution of step a0) comprises NaOH in a concentration of about 0.1 M to about 1.5 M, about 0.2 M to about 1.4 M, about 0.3 M to about 1.2 M, about 0.4 M to about 1.1 M, about 0.5 M to about 1 M. In one preferred embodiment, the alkaline solution of step a0) comprises NaOH in a concentration of about 0.9 M to about 1.1 M.

In certain embodiments of the above aspects and other embodiments, the alkaline solution of step a0) comprises NaOH in a concentration of at least about 0.05 M, at least about 0.1 M, at least about 0.2 M, at least about 0.3 M, at least about 0.4 M, at least about 0.5 M, at least about 0.6 M, at least about 0.7 M, at least about 0.8 M, in one preferred embodiment at least about 0.9 M, at least about 1 M, at least about 1.1 M, at least about 1.2 M, at least about 1.3 M, at least about 1.4 M, or at least about 1.5 M.

In certain embodiments of the above aspects and other embodiments, the pre-incubation is with an NaOH solution of 100 mM to 1.2 M. In certain embodiments of the above aspects and other embodiments, the pre-incubation is with an NaOH solution for 30 minutes to 4.5 hours. In one embodiment of the above aspects and other embodiments, the pre-incubation is with an at least 100 mM NaOH alkaline solution for at least about 30 minutes. In one preferred embodiment of the above aspects and other embodiments, the pre-incubation is with a 1 M NaOH alkaline solution for about 4 hours.

In certain embodiments of the above aspects and other embodiments, the pre-incubation is with at least 50 L, or 50 L to 200 L, or 100 L to 150 L of the alkaline solution per m² of depth filter area. In one preferred embodiment of the above aspects and other embodiments, the pre-incubation is with 100 L of the alkaline solution per m² of depth filter area (100 L/m²).

In certain embodiments of the above aspects and other embodiments, the alkaline regeneration solution of step b) is an alkaline aqueous buffered solution.

In certain embodiments of the above aspects and other embodiments, the alkaline solution of step a0) additionally comprises NaCl.

In certain embodiments of the above aspects and other embodiments, the alkaline solution of step a0) comprises NaCl in a concentration of about 0.5 M to about 2.5 M, about 0.6 M to about 2.3 M, about 0.7 M to about 2 M, about 0.8 M to about 1.8 M, or about 0.9 M to about 1.5 M. In one preferred embodiment, the alkaline solution of step a0) comprises NaOH in a concentration of about 0.9 M to about 1.1 M.

In certain embodiments of the above aspects and other embodiments, the alkaline solution of step a0) comprises NaCl in a concentration of at least about 0.5 M, at least about 0.7 M, at least about 0.8 M, at least about 0.9 M, or in one preferred embodiment at least about 1 M.

In certain embodiments of the above aspects and other embodiments, the alkaline regeneration solution of step a0) has a pH value of about 9 to 14, about 9.5 to 14, or about 10 to 14.

In certain embodiments of the above aspects and other embodiments, the alkaline regeneration solution of step a0) has a pH value of about 9 or more, about 9.5 or more, in one preferred embodiment about 10 or more, about 10.5 or more, or about 11 or more.

In certain embodiments of the above aspects and other embodiments, the aqueous buffered regeneration solution of step b) is the equilibration buffer/the buffer used for equilibrating the depth filter.

In certain embodiments of the above aspects and other embodiments, the equilibration buffer has a pH value of about 3.5 to about 8, or about 3.5 to about 6, or in a preferred embodiment about 4 to about 5.5. In one preferred embodiment, the equilibration buffer has a pH value of pH 4+/−0.2. In one preferred embodiment, the equilibration buffer has a pH value of pH 5.5+/−0.2.

In certain embodiments of the above aspects and other embodiments, the equilibration buffer comprises 150 mM acetic acid/tris (hydroxymethyl) amino methane.

Regeneration with Water and Alkaline Pre-Treatment

It has been found that water can be used a regeneration solution and that a pre-treatment of the depth filter with an alkaline solution has a beneficial effect.

One aspect according to the invention is a method for purifying a therapeutic polypeptide, characterized in that the method comprises the following steps:
a0) incubating a depth filter with an alkaline solution,
a) filtering an aqueous composition containing said therapeutic polypeptide and impurities through said depth filter (obtained in step a0)), recovering the flow-through and thereby obtaining said purified therapeutic polypeptide,
b) contacting said depth filter (after step a)) with water and thereby regenerating the depth filter,
and
c) repeating steps a) and b) one or more times.

One further aspect according to the invention is a method for producing a therapeutic polypeptide, characterized in that the method comprises the following steps:
a0) incubating a depth filter with an alkaline solution,
a) filtering an aqueous composition containing said therapeutic polypeptide and impurities through said depth filter (obtained in step a0)), recovering the flow-through and thereby obtaining said therapeutic polypeptide,
b) contacting said depth filter (after step a)) with water and thereby regenerating the depth filter,
and
c) repeating steps a) and b) one or more times.

In certain embodiments of the above aspects and other embodiments, the alkaline solution of step a0) is a solution comprising NaOH or KOH.

In certain embodiments of the above aspects and other embodiments, the alkaline solution of step a0) comprises NaOH in a concentration of about 0.1 M to about 1.5 M, about 0.2 M to about 1.4 M, about 0.3 M to about 1.2 M, about 0.4 M to about 1.1 M, about 0.5 M to about 1 M. In one preferred embodiment, the alkaline solution of step a0) comprises NaOH in a concentration of about 0.9 M to about 1.1 M.

In certain embodiments of the above aspects and other embodiments, the alkaline solution of step a0) comprises NaOH in a concentration of at least about 0.05 M, at least about 0.1 M, at least about 0.2 M, at least about 0.3 M, at least about 0.4 M, at least about 0.5 M, at least about 0.6 M, at least about 0.7 M, at least about 0.8 M, in one preferred embodiment at least about 0.9 M, at least about 1 M, at least about 1.1 M, at least about 1.2 M, at least about 1.3 M, at least about 1.4 M, or at least about 1.5 M.

In certain embodiments of the above aspects and other embodiments, the pre-incubation is with an NaOH solution of 100 mM to 1.2 M. In certain embodiments of the above aspects and other embodiments, the pre-incubation is with an NaOH solution for 30 minutes to 4.5 hours. In one embodiment of the above aspects and other embodiments, the pre-incubation is with an at least 100 mM NaOH alkaline solution for at least about 30 minutes. In one preferred embodiment of the above aspects and other embodiments, the pre-incubation is with a 1 M NaOH alkaline solution for about 4 hours.

In certain embodiments of the above aspects and other embodiments, the pre-incubation is with at least 50 L, or 50 L to 200 L, or 100 L to 150 L of the alkaline solution per m² of depth filter area. In one preferred embodiment of the above aspects and other embodiments, the pre-incubation is with 100 L of the alkaline solution per m² of depth filter area (100 L/m²).

In certain embodiments of the above aspects and other embodiments, the alkaline regeneration solution of step b) is an alkaline aqueous buffered solution.

In certain embodiments of the above aspects and other embodiments, the alkaline solution of step a0) additionally comprises NaCl.

In certain embodiments of the above aspects and other embodiments, the alkaline solution of step a0) comprises NaCl in a concentration of about 0.5 M to about 2.5 M, about 0.6 M to about 2.3 M, about 0.7 M to about 2 M, about 0.8 M to about 1.8 M, or about 0.9 M to about 1.5 M. In one preferred embodiment, the alkaline solution of step a0) comprises NaOH in a concentration of about 0.9 M to about 1.1 M.

In certain embodiments of the above aspects and other embodiments, the alkaline solution of step a0) comprises NaCl in a concentration of at least about 0.5 M, at least about 0.7 M, at least about 0.8 M, at least about 0.9 M, or in one preferred embodiment at least about 1 M.

In certain embodiments of the above aspects and other embodiments, the alkaline regeneration solution of step a0) has a pH value of about 9 to 14, about 9.5 to 14, or about 10 to 14.

In certain embodiments of the above aspects and other embodiments, the alkaline regeneration solution of step a0) has a pH value of about 9 or more, about 9.5 or more, in one preferred embodiment about 10 or more, about 10.5 or more, or about 11 or more.

One further aspect according to the invention is the use of an acidic solution for regeneration of a depth filter (comprising silica) that is being used at least two times in the purification of a therapeutic polypeptide.

One further aspect according to the invention is the use of an alkaline solution for regeneration of a depth filter (comprising silica) that is being used at least two times in the purification of a therapeutic polypeptide.

Effects and Uses of the Methods According to the Invention

In certain embodiments of all above aspects and embodiments, the method reduces the (HCP-based/originating) enzymatic hydrolytic activity/hydrolysis activity rate.

In certain embodiments of all above aspects and embodiments, the method reduces/removes HCPs (host cell proteins) with enzymatic hydrolytic activity.

In certain embodiments of all above aspects and embodiments of the invention, the method reduces the enzymatic hydrolysis activity rate in the produced/purified therapeutic polypeptide compared to the aqueous composition (of step a) prior to the application to the filter).

In certain embodiments of all above aspects and embodiments of the invention, the method reduces the enzymatic hydrolysis activity rate in the produced/purified therapeutic polypeptide compared to the aqueous composition (of step a) prior to the application to the filter).

In certain embodiments of all above aspects and embodiments of the invention, the method reduces the enzymatic hydrolysis activity rate in the produced/purified therapeutic polypeptide by at least 25%, or by at least 30%, or by at least 35%, or by at least 40% compared to the aqueous composition (of step a) prior to the application to the filter).

In certain embodiments of the above aspects and the other embodiments, the enzymatic hydrolysis activity rate is determined by a lipase activity assay (as described herein).

In certain embodiments of the above aspects and the other embodiments, enzymatic hydrolysis activity rate is determined by the enzymatic hydrolysis of a substrate. In certain embodiments of the above aspects and the other embodiments, enzymatic hydrolysis activity rate is determined by monitoring the conversion of a substrate. In certain embodiments of the above aspects and the other embodiments, enzymatic hydrolysis activity rate is determined by monitoring the conversion of a fluorogenic substrate ‘4-Methylumbelliferyl Caprylate’ (4-MU-C8) by cleavage of the ester bond by hydrolases present in the sample/the purified therapeutic polypeptide into a fluorescent moiety, i.e. 4-Methylumbelliferyl (4-MU).

In certain embodiments of all above aspects and embodiments, the enzymatic hydrolysis activity rate is determined by the enzymatic hydrolysis of a substrate. In one embodiment, the hydrolysis rate is the hydrolysis rate of 4-Methylumbelliferyl Caprylate or polysorbate (in one embodiment polysorbate 20).

In certain embodiments of all above aspects and embodiments, a pharmaceutical formulation comprising the purified/produced therapeutic polypeptide and a polysorbate shows reduced hydrolysis of the polysorbate compared to an identical pharmaceutical formulation comprising the aqueous composition instead of the purified/produced therapeutic polypeptide. In certain embodiments of all above aspects and embodiments of the invention, the yield of the (monomeric) therapeutic polypeptide obtained when using a depth filter after regeneration is at least 80%, or at least 85%, or at least 90%, or at least 95% of the yield obtained when using the depth filter for the first time, i.e. without regeneration/after the first filtration with the depth filter.

In certain embodiments of all above aspects and embodiments of the invention, the yield of the (monomeric) therapeutic polypeptide obtained using a depth filter after regeneration is at least 90% of the yield obtained when using the depth filter for the first time, i.e. without regeneration/after the first filtration with the depth filter.

In certain embodiments of all above aspects and embodiments of the invention, the depth filter comprises a substrate comprising one or more of a diatomaceous earth composition, a silica composition, a cellulose fiber, a polymeric fiber, a cohesive resin, or/and an ash composition.

In certain embodiments of all above aspects and embodiments of the invention, the depth filter comprises (a substrate comprising) one or more selected from a diatomaceous earth material (composition), a silica material (composition), a cellulose fiber, and a polymeric fiber.

In certain embodiments of all above aspects and embodiments of the invention, at least a portion of the substrate of the depth filter comprises a surface modification.

In certain embodiments of all above aspects and embodiments of the invention, at least a portion of the substrate of the depth filter comprises one or more surface modification(s) selected from a quaternary amine surface modification, charged surface group modifications (a cationic surface modifications or an anionic surface modifications). In one preferred embodiment, the surface modification is a cationic surface modification.

In certain embodiments of all above aspects and embodiments of the invention, the depth filter comprises a material that is selected from the group of
(i) polyacrylic fiber and silica;
(ii) cellulose fibers, diatomaceous earth, and perlite, and
(iii) cellulose fiber and charged surface groups (cationic charge modifications).

In certain embodiments of all above aspects and embodiments of the invention, the depth filter is selected from the group consisting of an X0SP depth filter (Millistak+® HC Pro X0SP), or a PDD1 depth filter (SUPRAcap™-50 (SC050PDD1)), or aVR02 depth filter (Zeta Plus™ Biocap VR02).

In certain embodiments of all above aspects and embodiments of the invention, the depth filter is an X0SP depth filter (Millistak+® HC Pro X0SP), or a PDD1 depth filter (SUPRAcap™-50 (SC050PDD1)).

In certain embodiments of all above aspects and embodiments of the invention, the depth filter is contacted with the regeneration solution for about 20 min. or more, about 30 min. or more, about 40 min. or more, about 50 min. or more, or about 60 min. or more.

It is understood that the depth filter can be used before or after a first chromatography step. It can also be used before or after a second, third, fourth or any further chromatography step. In one preferred embodiment of all aspects and other embodiments according to the invention, the depth filter is before or after a first chromatography step performed with the aqueous composition. One especially preferred use is before the first chromatography step (i.e. after the harvesting of the cells from cell cultivation).

In certain embodiments of all above aspects and embodiments of the invention, the method according to the invention comprises as first step or as last step a chromatography step. In one preferred embodiment of all above aspects and embodiments of the invention, the method comprises as first step a chromatography step and the aqueous composition of step a) is the eluate (fraction) of the chromatography step comprising the therapeutic polypeptide.

In one preferred embodiment of all above aspects and embodiments of the invention, the method comprises as last step a chromatography step and the produced/purified therapeutic polypeptide obtained in step a) is used in the chromatography step/applied to the chromatography material.

In certain embodiments of the above aspects and the other embodiments, the therapeutic polypeptide is a recombinantly produced protein. In certain embodiments of the above aspects and the other embodiments, the therapeutic polypeptide is a recombinantly produced protein that is being formulated with a nonionic surfactant like e.g. a polysorbate. In certain embodiments of the above aspects and the other embodiments, the therapeutic polypeptide is selected from the group of therapeutic polypeptides consisting of antibodies, antibody fragments, antibody fusion polypeptides, Fc-region fusion polypeptides, interferons, blood factors, cytokines, proteins for vaccination, and enzymes.

### Definitions

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about” whether or not explicitly indicated. The term “about” denotes a range of +/−20% of the thereafter following numerical value. In one embodiment, the term about denotes a range of +/−10% of the thereafter following numerical value. In one embodiment the term about denotes a range of +/−5% of the thereafter following numerical value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass all sub ranges subsumed therein. For example, a range of “1 to 10” includes any and all sub ranges between (and including) the minimum value of 1 and the maximum value of 10, that is, any and all sub ranges having a minimum value of equal to or greater than 1 and a maximum value of equal to or less than 10, e.g., 5.5 to 10.

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N. Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “aqueous solution” or “aqueous composition”, as used herein, relates to any liquid preparation wherein the concentration of water (H₂O) in the solvent is at least 50% (w/v), in an embodiment at least 75% (w/v), in a further embodiment at least 90% (w/v), in a further embodiment at least 95% (w/v), in a further embodiment at least 98% (w/v), in a further embodiment at least 99% (w/v), in a further embodiment is at least 99.5% (w/v). Thus, the term aqueous solution encompasses liquid preparations comprising up to 50% (w/v) D20 or PEG (poly ethylene glycol).

Further, as used herein, the term “solution” indicates that at least a fraction of the compounds (solutes) in the solution is dissolved in the solvent. Methods for preparing solutions are known in the art. Thus, the term aqueous solution, in an embodiment, relates to a liquid preparation comprising a therapeutic polypeptide, which is, at least partially, dissolved in a solvent comprising, in an embodiment consisting of, a buffered solution.

The term “comprising” also encompasses the term “consisting of”.

Antibody Production and Purification

Depth filters can be used at various stages of monoclonal antibody (mAb) production/purification processes. Such processes can comprise one or more of the following steps in the following or a different order:
Harvest: Separates cells and cell debris from protein-containing supernatant. The harvest step is typically performed using centrifugation and/or filtration.
Fc-binding/Protein A affinity chromatography (capture step): This step captures mAb molecules by preferentially binding to the Fc region at neutral pH and allows the rest of the harvested supernatant to be removed. The mAb molecules can then eluted at low pH resulting in an affinity elution pool.
Viral inactivation: Incubation of the affinity elution pool at low pH can inactivate viruses.
Cation exchange chromatography: This step can remove HCPs, mAb aggregates and antibody fragments, and can comprise a bind-elute or flow-through step.
Anion exchange chromatography: This step can remove DNA, leached protein A and other trace contaminants, and can be performed in bind-elute or flow-through.
Viral filtration: Single-pass (dead-end) filtration with membranes designed to remove viruses.
Ultrafiltration: In this step, by passing the sample through a semi-permeable membrane (pore sizes may range between 0.1-0.01 μm), the mAb molecules can be concentrated further. If this is the final purification step, the elution buffer can be exchanged to a final formulation buffer.

Depth filtration can be used, for example, prior to or after viral inactivation, ion exchange chromatography, viral filtration, or/and ultrafiltration. Depth filtration can be used to reduce (process-related) impurities. Depth filtration can be used to reduce (process-related) impurities that are hydrolytically active or possess hydrolytic activity. In other words, depth filtration can reduce the enzymatic hydrolysis activity rate. This effect/rate can be determined/measured by methods known to the skilled person, some of which are described herein (e.g. Lipase activity assay (LEAP) in Example 21). In some embodiments, depth filtration is used to reduce the hydrolytic activity of an aqueous composition. In some embodiments, depth filtration is used to reduce host cell DNA in an aqueous solution.

Depth filtration can also be used in further downstream stages of the purification process for secondary clarification and haze removal, and for further removal of process-related impurities as disclosed herein.

As used herein the term “depth filter” denotes a filter that achieves filtration, i.e. separation of material, within the depth of the filter material. In some embodiments, the depth filter comprises a porous filtration medium capable of retaining portions of a sample, such as, e.g., cell components and debris, wherein filtration occurs, e.g., within the depth of the filter material. A common class of such filters is those that comprise a (random) matrix of bonded fibers (or otherwise fixed), to form a complex, tortuous maze of flow channels. Particle separation in these filters generally results from entrapment by or adsorption to the filter material. Frequently used depth filter media for bioprocessing of cell culture broths and other feedstocks consists of cellulose fibers (as matrix) and a filter aid such as diatomaceous earth (DE). Another depth filter used in the context of the invention is a depth filter comprising silica and polyacrylic fiber. In some embodiments, the depth filter is a synthetic filter. In some embodiments, the depth filter comprises a silica filter aid, and/or polyacrylic fiber. In some embodiments, the depth filter comprises a silica filter aid, and/or polyacrylic fiber, and/or non-woven material. In some embodiments, the depth filter comprises silica and polyacrylic fiber as non-woven material. Depth filter media, unlike absolute filters, retain particles and other impurities throughout the porous media allowing, e.g., for retention of particles both larger and smaller than the pore size. Particle and impurity retention is thought to involve size exclusion and adsorption through hydrophobic, ionic and other interactions. Depth filters are advantageous because they remove contaminants/impurities. The depth filter may be a multi-layer depth filter comprising multiple levels of depth filter media, which are layered in series. Employing multiple depth filters ensures that more of the filtrate stream efficiently contacts the depth filter media, enabling a better adsorption profile for the impurities.

In some embodiments, the depth filter comprises synthetic material, non-synthetic material, or a combination thereof. In some embodiments, the depth filter comprises a substrate comprising one or more of a diatomaceous earth composition, a silica composition, a cellulose fiber, a polymeric fiber, a cohesive resin, and an ash composition. In some embodiments, the depth filter is selected from the group consisting of an X0SP depth filter (Millistak+® HC Pro X0SP), a PDD1 depth filter (Pall/3M PDD1 SUPRAcap™-50 (SC050PDD1)), or a VR02 depth filter (Zeta Plus™ Biocap VR02).

In some embodiments, the depth filter comprises cellulose fibers, diatomaceous earth, and perlite. In some embodiments, the depth filter comprises two layers, wherein each layer comprises a cellulose filter matrix, and wherein the cellulose filter matrix is impregnated with a filter aid comprising one or more of diatomaceous earth or perlite. In some embodiments, the depth filter comprises two layers, wherein each layer comprises a cellulose filter matrix, wherein the cellulose filter matrix is impregnated with a filter aid comprising one or more of diatomaceous earth or perlite, and wherein each layer further comprises a resin binder. In some embodiments, the depth filter is a PDD1 depth filter.

In some embodiments, the depth filter comprises a silica, such as a silica filter aid, and a polyacrylic fiber. In some embodiments, the depth filter comprises two layers of filter media, wherein a first layer comprises a silica, such as a silica filter aid, and a second layer comprises a polyacrylic fiber, such as a polyacrylic fiber pulp. In some embodiments, the depth filter is a depth filter comprising synthetic material and does not comprise diatomaceous earth and/or perlite. In some embodiments, the depth filter is a X0SP depth filter.

In some embodiments, the depth filter comprises cellulose fibers (as matrix) and charged surface groups (ionic charge modifications). In some embodiments, the depth filter comprises cellulose fibers (as matrix) and a cationic charge modifier that is chemically bound to the matrix components. In some embodiments, the depth filter is a VR02 depth filter.

In some embodiments, the silica filter aid is a precipitated silica filter aid. In some embodiments, the filter aid is an aspect of the filter, such as a layer, that aids with performing the filter function. In some embodiments, the silica filter aid is a silica gel filter aid. In some embodiments, the depth filter has a pore size of about 0.05 μm to about 0.2 μm, such as about 0.1 μm. In some embodiments, the depth filter has a surface area in the range of about 0.1 m² to about 1.5 m², such as at least about 22 cm², at least about 23 cm², or at least about 25 cm², at least about 0.11 m², at least about 0.55 m², or of at least about 1.1 m² or greater.

It is understood that depth filters have a certain capacity (binding capacity). This binding capacity defines the upper limit of the amount of therapeutic polypeptide molecules per surface that can be applied to the filter without impairing the filter properties (separation efficiency and yield). The skilled person knows how to determine said binding capacity limit for each depth filter and for each molecule. This can be done using standard methods.

When exceeding the capacity limit of a given depth filter, the ability of the filter to effectively remove impurities from the load and, thus, for example, the obtainable yield and/or the purity of the molecule of interest will decrease.

The skilled person understands that the depth filter functions well until reaching its binding capacity limit and there will be no need for regenerating of the depth filter beforehand.

The term “incubating” in connection with the pre-treatment of a depth filter prior to its first use includes different types of contact of the solution used for pre-treatment with the depth filter. This can for example be in the form of contacting the depth filter with a solution by flowing the solution through the depth filter for a period of time, i.e. washing the depth filter with the solution with a certain flow rate (e.g. 7 or 10 mL/min). The depth filter can also be contacted by placing it into a (pre-treatment) solution and storing it in the solution for a certain time. It is also possible that the flow-through of the solution/washing is performed first, followed by a pausing of the flow and therefore resting the depth filter in the solution; or vice-versa (i.e. storing before and/or after washing).

The invention encompasses the purification and production of therapeutic polypeptides. The therapeutic polypeptide can be of different nature. The therapeutic polypeptide is designed and suitable for therapeutic as well as for diagnostic purposes. In certain embodiments of the above aspects and the other embodiments, the therapeutic polypeptide is a recombinantly produced protein. In certain embodiments of the above aspects and the other embodiments, the therapeutic polypeptide is a recombinantly produced protein that is being formulated with a nonionic surfactant like e.g. a polysorbate. In certain embodiments of the above aspects and the other embodiments, the therapeutic polypeptide is selected from the group of therapeutic polypeptides consisting of antibodies, antibody fragments, antibody fusion polypeptides, Fc-region fusion polypeptides, interferons, blood factors, cytokines, proteins for vaccination, and enzymes. In a preferred embodiment of the above aspects and the other embodiments, the therapeutic polypeptide is an antibody.

The term “antibody” includes full-length antibodies and antigen-binding fragments thereof. In some embodiments, a full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain). In some embodiments, the antibody is a monoclonal antibody. In some embodiments, the antibody is a human, humanized, or chimeric antibody. In some embodiments, the antibody is a chimeric antibody. In some embodiments, the antibody is a semi-synthetic antibody. In some embodiments, the antibody is a diabody. In some embodiments, the antibody is a humanized antibody. In some embodiments, the antibody is a multispecific antibody, such as a bispecific antibody. In some embodiments, the antibody is a bispecific antibody. In some embodiments, the antibody is linked to a fusion protein. In some embodiments the antibody is linked to an immunostimulating protein, such as an interleukin. In some embodiments the antibody is linked to a protein which facilitates the entry across the blood brain barrier.

The term “multispecific antibodies” as used herein refer to monoclonal antibodies that have binding specificities for at least two different sites, i.e., different epitopes on different antigens or different epitopes on the same antigen. In certain aspects, the multispecific antibody has two binding specificities (bispecific antibody). In certain aspects, the multispecific antibody has three or more binding specificities. Multispecific antibodies may be prepared as full length antibodies or antibody fragments.

The term “semi-synthetic” in reference to an antibody or antibody moiety means that the antibody or antibody moiety has one or more naturally occurring sequences and one or more non-naturally occurring (i.e., synthetic) sequences.

The following Examples and Figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Reference values (no regeneration) for hydrolytic activity measured by LEAP (see Examples 15, 21); Filter: PDD1; Therapeutic polypeptide: Crovalimab; Marker (thin vertical line): mass throughput of 1320 g/m² (binding capacity limit).

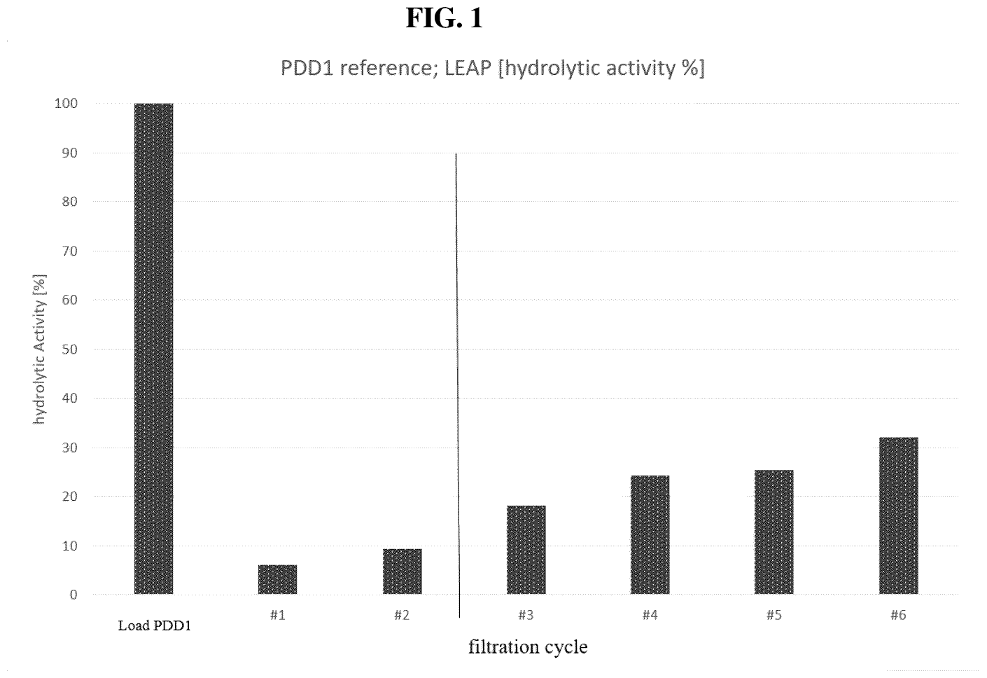

FIG. 2 Hydrolytic activity measured by LEAP for three different settings: 1) grey dotted: Application of water and buffer between the filtration cycles (see Example 9), 2) black dotted: pre-treatment and application of an alkaline solution between the filtration cycles (see Example 10), 3) grey: Application of an acidic solution (see Example 11); Filter: PDD1; Therapeutic polypeptide: Crovalimab; Marker (thin vertical line): mass throughput of 1320 g/m² (binding capacity limit).

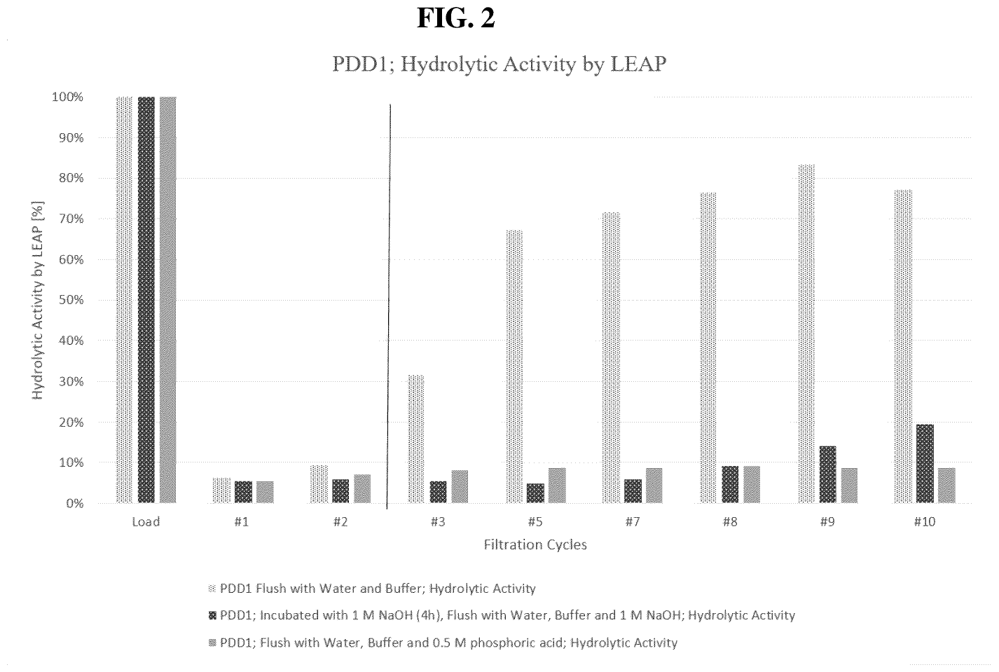

FIG. 3 Yield of mainpeak/main product for three different settings: 1) grey dotted: Application of water and buffer between the filtration cycles (see Example 9), 2) black dotted: pre-treatment and application of an alkaline solution between the filtration cycles (see Example 10), 3) grey: Application of an acidic solution (see Example 11); Filter: PDD1; Therapeutic polypeptide: Crovalimab.

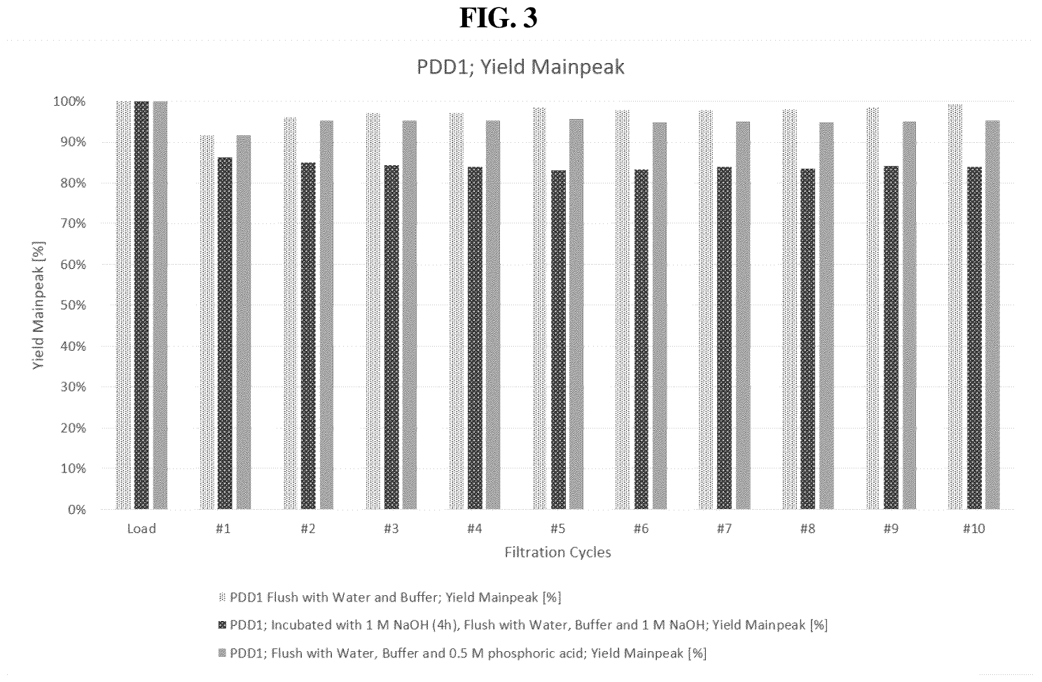

FIG. 4 Reference values (no regeneration) for hydrolytic activity measured by LEAP (see Examples 12, 21); Filter: X0SP; Therapeutic polypeptide: Crovalimab; Marker (thin vertical line): mass throughput of 2640 g/m² (binding capacity limit).

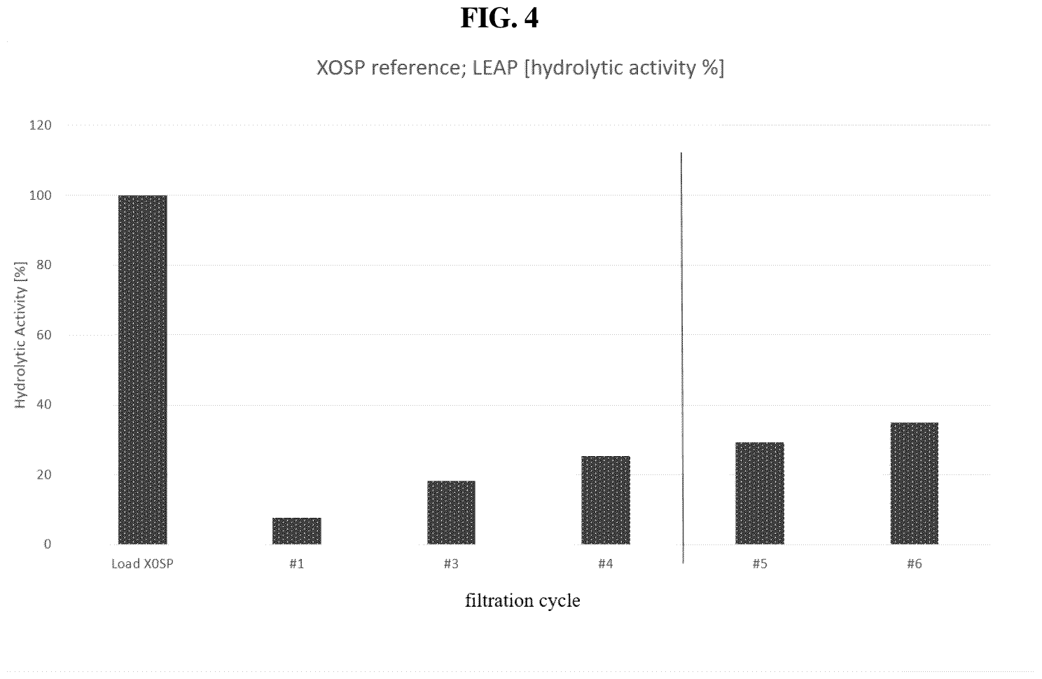

FIG. 5 Hydrolytic activity measured by LEAP for three different settings: 1) grey dotted: Application of water and buffer between the filtration cycles (see Example 6), 2) black dotted: pre-treatment and application of an alkaline solution between the regeneration cycles (see Example 7), 3) grey: Application of an acidic solution between the filtration cycles (see Example 8); Filter: X0SP; Therapeutic polypeptide: Crovalimab; Marker (thin vertical line): mass throughput of 2640 g/m² (binding capacity limit).

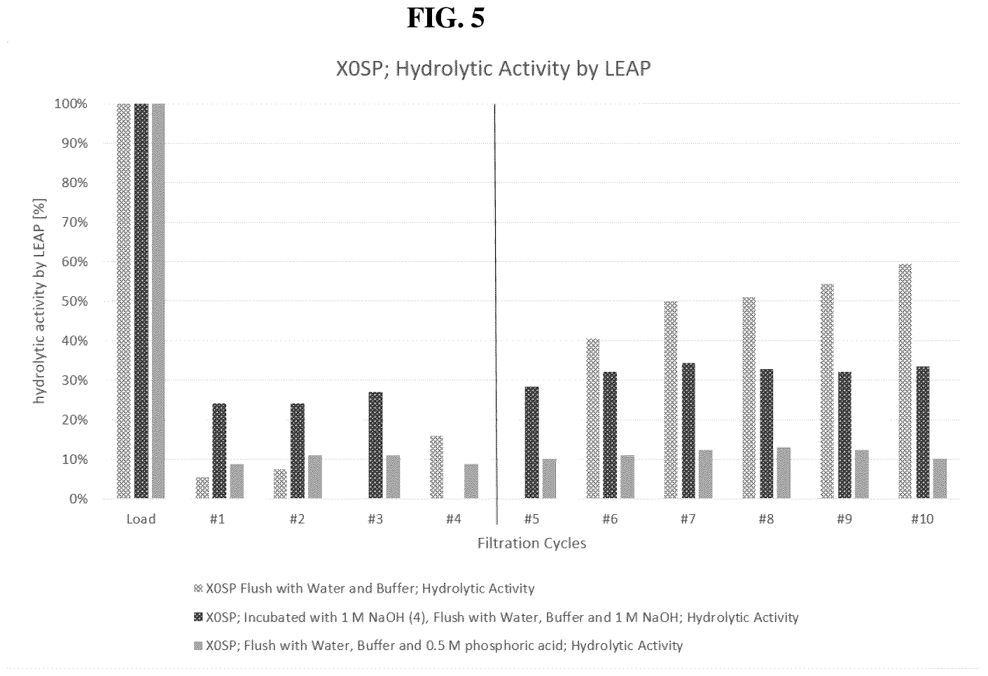

FIG. 6 Yield of mainpeak/main product for three different settings: 1) grey dotted: Application of water and buffer between the filtration cycles (see Example 6), 2) black dotted: pre-treatment and application of an alkaline solution between the regeneration cycles (see Example 7), 3) grey: Application of an acidic solution between the filtration cycles (see Example 8); Filter: X0SP; Therapeutic polypeptide: Crovalimab.

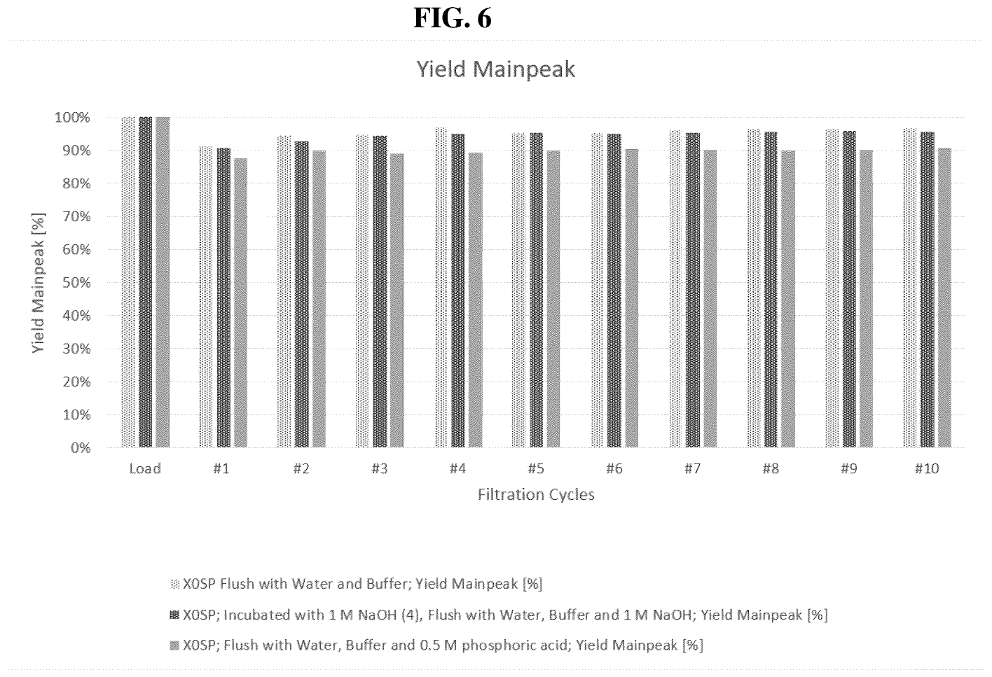

FIG. 7 Reference values (no regeneration) for hydrolytic activity measured by LEAP (see Examples 18, 21); Filter: VR02; Therapeutic polypeptide: Crovalimab; Marker (thin vertical line): mass throughput of 660 g/m² (binding capacity limit).

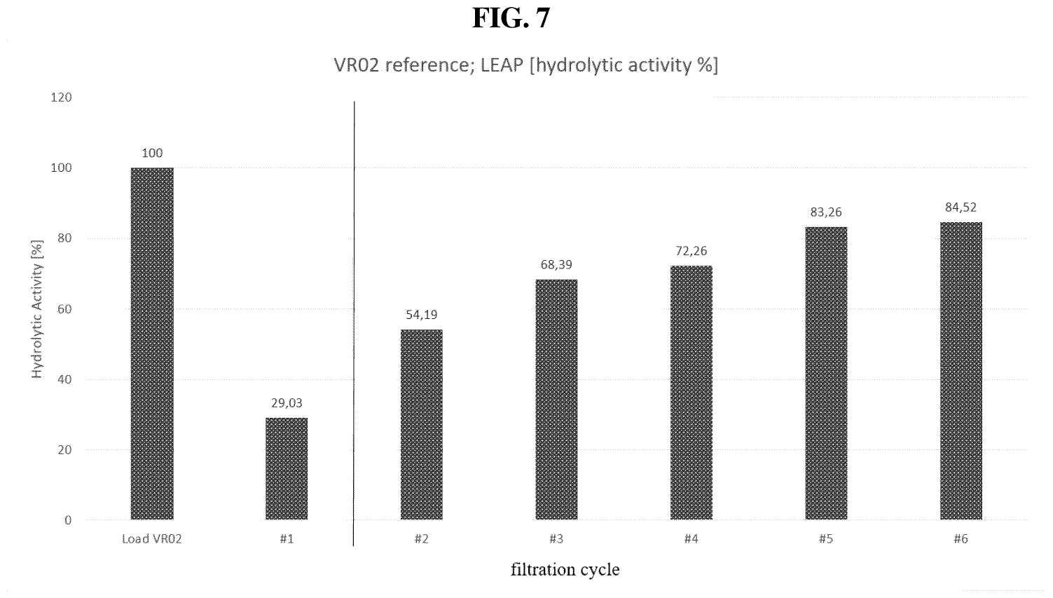

FIG. 8 Hydrolytic activity measured by LEAP for two different settings: 1) grey dotted: Application of water and buffer between the filtration cycles (see Example 19), 2) black dotted Application of an acidic solution between the filtration cycles (see Example 20); Filter: VR02; Therapeutic polypeptide: Crovalimab; Marker (thin vertical line): mass throughput of 660 g/m² (binding capacity limit)

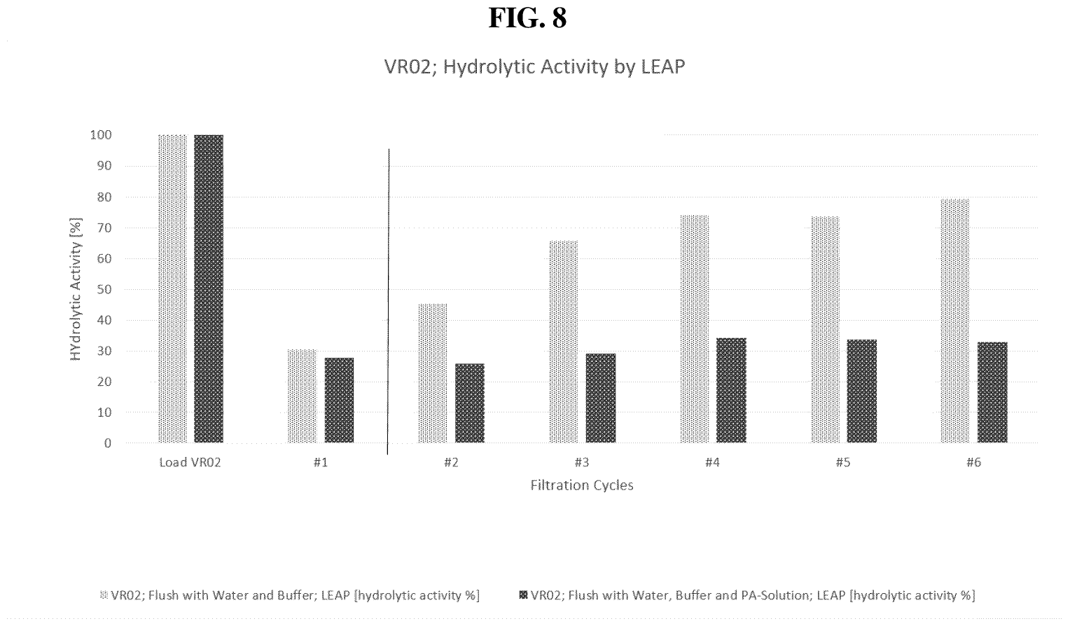

FIG. 9 Yield of mainpeak/main product for two different settings: 1) grey dotted: Application of water and buffer between the filtration cycles (see Example 19), 2) black dotted Application of an acidic solution between the filtration cycles (see Example 20); Filter: VR02; Therapeutic polypeptide: Crovalimab.

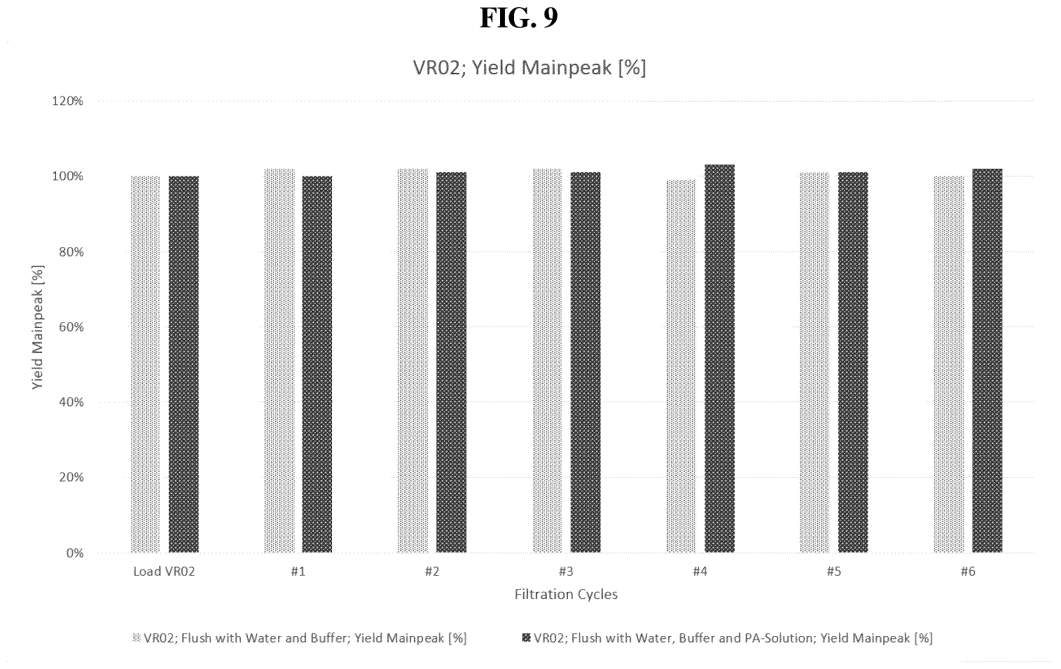

### EXAMPLES

### Overview

| Example | antibody | Filter type | Regeneration |
|---|---|---|---|
| 1 | bispecific anti-GPRC5D | X0SP | 1M NaCl, 0.5M NaOH |
| | antibody | | (no pre-treatment) |
| 2 | bispecific anti-CD20/TfR | X0SP | 1M NaCl, 0.5M NaOH |
| | antibody | | (no pre-treatment) |
| 3 | bispecific anti-CD20/TfR | X0SP | 167 mM acetic acid, 300 |
| | antibody | | mM phosphoric acid (no |
| | | | pre-treatment) |
| 4 | antibody-IL2 fusion | X0SP | 167 mM acetic acid, 300 |
| | polypeptide | | mM phosphoric acid (no |
| | | | pre-treatment) |
| 5 | antibody-IL2 fusion | PDD1 | 167 mM acetic acid, 300 |
| | polypeptide | | mM phosphoric acid (no |
| | | | pre-treatment) |
| 6 | anti-C5 antibody | X0SP | Water and Buffer (no pre- |
| | | | treatment) |
| 7 | anti-C5 antibody | X0SP | 1M NaOH for pre- |
| | | | treatment and regeneration |
| 8 | anti-C5 antibody | X0SP | 0.5M phosphoric acid (no |
| | | | pre-treatment) |
| 9 | anti-C5 antibody | PDD1 | Water and Buffer (no pre- |
| | | | treatment) |
| 10 | anti-C5 antibody | PDD1 | 1M NaOH for pre- |
| | | | treatment and regeneration |
| 11 | anti-C5 antibody | PDD1 | 0.5M phosphoric acid (no |
| | | | pre-treatment) |
| 12 | anti-C5 antibody | X0SP | without (reference) |
| 13 | anti-C5 antibody | X0SP | 167 mM acetic acid, 300 |
| | | | mM phosphoric acid (no |
| | | | pre-treatment |
| 14 | anti-C5 antibody | X0SP | 1M NaOH pre-treatment, |
| | | | Water and Buffer |
| | | | regeneration |
| 15 | anti-C5 antibody | PDD1 | without (reference) |
| 16 | anti-C5 antibody | PDD1 | 167 mM acetic acid, 300 |
| | | | mM phosphoric acid (no |
| | | | pre-treatment) |
| 17 | anti-C5 antibody | PDD1 | 167 mM acetic acid, 300 |
| | | | mM phosphoric acid, |
| | | | Buffer (w/o water flush; |
| | | | no pre-treatment) |
| 18 | anti-C5 antibody | VR02 | without (reference) |
| 19 | anti-C5 antibody | VR02 | Water and Buffer (no pre- |
| | | | treatment) |
| 20 | anti-C5 antibody | VR02 | 167 mM acetic acid, 300 |
| | | | mM phosphoric acid (no |
| | | | pre-treatment) |

Materials and Methods

Antibodies:

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered and referred to according to numbering according to Kabat (Kabat, E. A., et al., Sequences of Proteins of Immunological Interest, 5th ed., Public Health Service, National Institutes of Health, Bethesda, MD (1991)).

The current invention is exemplified using a number of exemplary antibodies, including: an anti-CD20/TfR bispecific antibody as reported in WO 2017/055542 and therein in SEQ ID NO: 01 to 03 and 10; an anti-C5 antibody (Crovalimab) as reported in WO2017/104779 and therein in SEQ ID NO: 106 to 111; a fusion protein comprising an inert antibody conjugated at the C-terminus of the heavy chain to a human IL-2 (interleukin 2) as reported in WO2015/118016 and therein in SEQ ID NO: 19 and 50; an anti-GPRC5D/anti-CD3 bispecific antibody in 2+1 format as reported in WO2021/018859A2.

Synthetic Depth Filter Media:

Herein the synthetic depth filter Millistak+® HC Pro X0SP is used. It is commercially available from MilliporeSigma (Bedford, MA). The X0SP depth filter has a nominal pore size rating of 0.1 microns and is intended for secondary clarification applications.

A cellulose/diatomaceous earth-comprising (also containing silica) PDD1 depth filter (Pall PDD1 SUPRAcap™-50 (SC050PDD1)), was also used.

A cellulose-comprising (also containing silica) VR02 depth filter (Zeta Plus™ Biocap VR02) was also used.

Depth Filtration Device:

All testing was performed using 22 cm2, 23 cm² or 25 cm2 μPod1 scale devices.

The depth filter devices were fabricated using two layers of the depth filtration media encapsulated in single-use, over-molded device housing.

Recombinant DNA Techniques:

Standard methods were used to manipulate DNA as described in Sambrook, J. et al., Molecular Cloning: A laboratory manual; Cold Spring Harbor Laboratory Press, Cold Spring Harbor, New York, 1989. The molecular biological reagents were used according to the manufacturer's instructions.

Gene Synthesis:

Desired gene segments were prepared from oligonucleotides made by chemical synthesis. The long gene segments, which were flanked by singular restriction endonuclease cleavage sites, were assembled by annealing and ligating oligonucleotides including PCR amplification and subsequently cloned via the indicated restriction sites. The DNA sequences of the subcloned gene fragments were confirmed by DNA sequencing. Gene synthesis fragments were ordered according to given specifications at Geneart (Regensburg, Germany).

DNA Sequence Determination:

DNA sequences were determined by double strand sequencing performed at MediGenomix GmbH (Martinsried, Germany) or SequiServe GmbH (Vaterstetten, Germany).

DNA and protein sequence analysis and sequence data management: The GCG's (Genetics Computer Group, Madison, Wisconsin) software package version 10.2 and Infomax's Vector NT1 Advance suite version 8.0 was used for sequence creation, mapping, analysis, annotation and illustration.

Expression Vectors:

For the expression of the described bispecific antibodies, expression plasmids for transient expression (e.g. in HEK293 cells) based either on a cDNA organization with or without a CMV-intron A promoter or on a genomic organization with a CMV promoter can be applied.

Beside the antibody expression cassette the vectors contain:
an origin of replication which allows replication of this plasmid in E. coli, and
a ß-lactamase gene which confers ampicillin resistance in E. coli.

The transcription unit of the antibody gene is composed of the following elements:
unique restriction site(s) at the 5′ end
the immediate early enhancer and promoter from the human cytomegalovirus,
the intron A sequence in the case of cDNA organization,
a 5′-untranslated region derived from a human antibody gene,
an immunoglobulin heavy chain signal sequence,
the respective antibody chain encoding nucleic acid either as cDNA or with genomic exon-intron organization,
a 3′ untranslated region with a polyadenylation signal sequence,
a terminator sequence, and
unique restriction site(s) at the 3′ end.

The fusion genes encoding the antibody chains are generated by PCR and/or gene synthesis and assembled by known recombinant methods and techniques by connection of the according nucleic acid segments e.g. using unique restriction sites in the respective vectors. The subcloned nucleic acid sequences are verified by DNA sequencing. For transient transfections larger quantities of the plasmids are prepared by plasmid preparation from transformed E. coli cultures (Nucleobond AX, Macherey-Nagel).

For all constructs knob-into-hole heterodimerization technology was used with a typical knob (T366W) substitution in the first CH3 domain and the corresponding hole substitutions (T366S, L368A and Y407V) in the second CH3 domain (as well as two additional introduced cysteine residues S354C/Y349′C) (contained in the respective corresponding heavy chain (HC) sequences depicted above).

Cell Culture Techniques:

Standard cell culture techniques as described in Current Protocols in Cell Biology (2000), Bonifacino, J. S., Dasso, M., Harford, J. B., Lippincott-Schwartz, J. and Yamada, K. M. (eds.), John Wiley & Sons, Inc., are used.

Transient Transfections in HEK293 System:

The bispecific antibodies are produced by transient expression. Therefore a transfection with the respective plasmids using the HEK293 system (Invitrogen) according to the manufacturer's instruction is done. Briefly, HEK293 cells (Invitrogen) growing in suspension either in a shake flask or in a stirred fermenter in serum-free FreeStyle™ 293 expression medium (Invitrogen) are transfected with a mix of the respective expression plasmids and 293Fectin™ or fectin (Invitrogen). For 2 L shake flask (Corning) HEK293 cells are seeded at a density of 1.0*10⁶ cells/mL in 600 mL and incubated at 120 rpm, 8% CO₂. On the next day the cells are transfected at a cell density of approx. 1.5*10⁶ cells/mL with approx. 42 mL of a mixture of A) 20 mL Opti-MEM medium (Invitrogen) comprising 600 μg total plasmid DNA (1 μg/mL) and B) 20 ml Opti-MEM medium supplemented with 1.2 mL 293 fectin or fectin (2 μl/mL). According to the glucose consumption glucose solution is added during the course of the fermentation. The supernatant containing the secreted antibody is harvested after 5-10 days and antibodies are either directly purified from the supernatant or the supernatant is frozen and stored.

Optical Density Determination:

The protein concentration of purified antibodies and derivatives was determined by determining the optical density (OD) at 280 nm, using the molar extinction coefficient calculated on the basis of the amino acid sequence according to Pace, et al., Protein Science 4 (1995) 2411-1423.

Protein Concentration Determination (yield):

Photometric Determination:

Protein concentrations were determined by UV spectroscopy using a Cary® 50 UV-Vis Spectrophotometer (Varian). Protein samples were diluted in their respective buffers and measured as duplicates. Concentrations were determined according to the following equation deriving from Lambert-Beer law: c=(A_{280 nm}-A_{320 nm})/ε·d. F with c protein concentration [mg/ml], A absorbance, ε extinction coefficient [ml/(mg·cm)], d cell length [cm] and F dilution factor. The anti-C5 antibody-specific extinction coefficient is 1.44 ml/(mg·cm), the bispecific anti-GPRC5D antibody specific extinction coefficient is 1.43 ml/(mg·cm), the bispecific anti-CD20/TfR specific extinction coefficient is 1.57 ml/(mg·cm) and the antibody-IL2 fusion polypeptide specific extinction coefficient is 1.25 ml/(mg·cm).

Chromatographic Determination:

The concentration of the antibodies was quantitatively measured by affinity HPLC chromatography. Briefly, solution containing antibodies that bind to protein A are applied, e.g., to an Applied Biosystems Poros A/20 column in 200 mM KH₂PO₄, 100 mM sodium citrate, pH 7.4 and eluted with 200 mM NaCl, 100 mM citric acid, pH 2.5 on an Agilent HPLC 1100 system. The eluted antibody is quantified by UV absorbance and integration of peak areas. A purified standard IgG1 antibody served as a standard.

ELISA Determination:

Alternatively, the concentration of antibodies and derivatives in solutions is measured by Sandwich-IgG-ELISA. Briefly, StreptaWell High Bind Streptavidin A-96 well microtiter plates (Roche Diagnostics GmbH, Mannheim, Germany) are coated with 100 μL/well biotinylated anti-human IgG capture molecule F(ab′)2<h-FcT>BI (Dianova) at 0.1 μg/mL for 1 hour at room temperature or alternatively overnight at 4° C. and subsequently washed three times with 200 μL/well PBS, 0.05% Tween (PBST, Sigma). Thereafter, 100 L/well of a dilution series in PBS (Sigma) of the respective antibody containing solution is added to the wells and incubated for 1-2 hour on a shaker at room temperature. The wells are washed three times with 200 μL/well PBST and bound antibody is detected with 100 μl F(ab′)2<hFcγ>POD (Dianova) at 0.1 μg/mL as the detection antibody by incubation for 1-2 hours on a shaker at room temperature. Unbound detection antibody is removed by washing three times with 200 μL/well PBST. The bound detection antibody is detected by addition of 100 μL ABTS/well followed by incubation. Determination of absorbance is performed on a Tecan Fluor Spectrometer at a measurement wavelength of 405 nm (reference wavelength 492 nm).

Preparative Antibody Purification:

Antibodies were purified from filtered cell culture supernatants referring to standard protocols. In brief, antibodies were applied to a protein A Mab Select SuRe column (GE healthcare) and washed with buffer. Elution of antibodies was achieved at low pH followed by immediate neutralization. Antibody fractions were pooled, frozen and stored at −20° C., −40° C. or −80° C.

Hydrolytic Activity Determination—Lipase Activity Assay (LEAP):

The lipase activity was determined by monitoring the conversion of a substrate, such as a nonfluorescent substrate, to a detectable product of the hydrolytic enzyme, such as a fluorescent product. An exemplary method is described, e.g., in WO 2018/035025, which is hereby incorporated by reference in its entirety.

In more detail, with the LEAP assay hydrolase activity in samples was determined.

This was done by monitoring the conversion of a fluorogenic substrate ‘4-Methylumbelliferyl Caprylate’ (4-MU-C8, available from Chem Impex Int'l Inc Art. Nr. 01552) by cleavage of the ester bond by hydrolases present in the sample into a fluorescent moiety, i.e. 4-Methylumbelliferyl (4-MU). Cleaved 4-MU-C8, i.e. 4-MU, was excited with a light of wavelength 355 nm. The emitted radiation at a different wavelength of 460 nm was recorded on Tecan Infinite® 200 PRO device as readout. The determination was performed at 37° C. for 2 hours with recording every 10 mins to calculate the rate of substrate hydrolysis.

The sample to be analyzed was at first buffer exchanged to 150 mM Tris-Cl, pH 8.0, by using Amicon Ultra-0.5 ml centrifugal filter units (10,000 Da cut-off, Merck Millipore, Art. Nr. UFC501096). The assay reaction mixture constituted of 80 μL reaction buffer (150 mM Tris-Cl, pH 8.0, 0.25% (w/v) Triton X-100, 0.125% (w/v) Gum Arabic), 10 μL 4-MU-C8 substrate solution (1 mM in DMSO), and 10 μL protein containing sample. The protein samples' concentration were adjusted to be in the range between 1-30 g/L and 2-3 dilution series were performed for each determination. Each reaction was set up at least in duplicates in 96-well half-area polystyrene plates (black with lid and clear flat bottom, Corning Incorporated Art. Nr. 3882).

Host Cell Protein (CHOP) Determination:

The residual CHO HCP content in process samples is determined by an electrochemiluminescence immunoassay (ECLIA) on cobas e 411 immunoassay analyzer (Roche Diagnostics).

The assay is based on a sandwich principle using polyclonal anti-CHO HCP antibody from sheep.

First incubation: Chinese hamster ovary host cell protein (CHO HCP) from 15 μL sample (neat and/or diluted) and a biotin conjugated polyclonal CHO HCP specific antibody form a sandwich complex, which becomes bound to streptavidin-coated microparticles via interaction of biotin with streptavidin.

Second incubation: After addition of polyclonal CHO HCP-specific antibody labeled with ruthenium complex (Tris(2,2′-bipyridyl)ruthenium(II)-complex) a ternary sandwich complex is formed on the microparticles.

The reaction mixture is aspirated into the measuring cell where the microparticles are magnetically captured onto the surface of the electrode. Unbound substances are then removed in a washing step. Application of a voltage to the electrode then induces chemiluminescent emission which is measured by a photomultiplier.

The concentration of CHO HCP in the test sample is finally calculated from a CHO HCP standard curve of known concentration.

Host Cell DNA Determination:

The residual Chinese Hamster Ovary (CHO) deoxyribonucleic acid (DNA) in process samples is determined on FLOW FLEX System (Roche Diagnostics GmbH, Mannheim, Germany).

The FLOW FLEX System consists of three modules: FLOW PCR SETUP Instrument, MagNA Pure 96 Instrument and LightCycler®480.

The FLOW PCR SETUP Instrument module is used as PSH (FLOW Primary Sample Handling) for sample transfer from primary tubes into a 96 well processing plate, and as PSU (FLOW PCR SETUP Instrument) for transfer of extracted DNA from the 96 well output plate into the PCR plate.

The MagNA Pure 96 Instrument module is used for automated isolation of nucleic acids. To release the DNA, the sample material is incubated under denaturing conditions. The released DNA is separated from the other buffer and sample components by binding to magnetic glass particles via a magnet, and the bound DNA is then eluted with buffer. Up to 96 samples can be processed simultaneously.

The LightCycler®480 module (microplate LightCycler®) is used for quantification of DNA or RNA based on PCR technology. The Residual DNA CHO Kit uses specific PCR of highly conserved regions within the DNA of CHO. The highly specific forward primers and reverse primers bind specifically to the ends of the target sequence of single-stranded DNA. The CHO DNA probe, labeled with a fluorescent reporter dye (FAM) at the 5′ end and a quencher dye at the 3′ end, hybridizes between the primers and the target sequence of single-stranded DNA. As long as the probe is intact, the proximity of the Quencher dye suppresses the fluorescence of the reporter dye. Upon amplification, the Taq polymerase, due to its 5′→3′exonuclease activity, disrupts the probe attached to the target sequence. This releases the reporter dye and the fluorescence increases. The increase in fluorescence is directly proportional to the amount of PCR product. The amount of CHO DNA in the samples is quantified with a standard curve.

Size Exclusion High Performance Liquid Chromatography (SE-HPLC):

Size exclusion chromatography (SEC) for the determination of the aggregation and oligomeric state of antibodies was performed by HPLC chromatography. Briefly, protein A purified antibodies were applied to a Tosoh TSK-Gel G3000SWXL (7.8×300 mm; 5 μm (TOSOH Bioscience Nr. 08541)) in 250 mM KCl, 200 mM KH₂PO₄/K₂HPO₄ buffer (pH 7.0) on an Dionex Ultimate® system (Thermo Fischer Scientific). The eluted antibody was quantified by UV absorbance and integration of peak areas. BioRad Gel Filtration Standard 151-1901 served as a standard.

MCE (Caliper):

Purity and antibody integrity were analyzed by CE-SDS using microfluidic Labchip technology (PerkinElmer, USA). Therefore, 5 μl of antibody solution was prepared for CE-SDS analysis using the HT Protein Express Reagent Kit according manufacturer's instructions and analyzed on LabChip GXII system using a HT Protein Express Chip. Data were analyzed using LabChip GX Software.

### Example 1

Filtration of a T-Cell Bispecific Anti-GPRC5D Antibody Solution with a Silica-Containing Millipore Millistak+® HC Pro X0SP Filter, Regenerated with Alkaline Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Millipore Millistak+® HC Pro X0SP filter with 23 cm² filter area; Lot.: CP8MA89804
3) equilibration buffer: 150 mM acetic acid/tris, adjusted to pH 5.5 in purified water type II (Millipore Super Q)
4) alkaline regeneration solution: 1 M NaCl, 0.5 M NaOH, pH 12.6

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

Experimental Setup:

Following steps were performed sequentially:
1) An intermediate “Affinity Pool pH 5.5” containing the T-cell bispecific antibody was applied to the conditioned 23 cm²×0SP filter unit. The mass throughput was 600 g/m². The corresponding calculated volume throughput was 52.17 L/m². The feed flow was adjusted to 200 LMH (liter per square meter per hour; L*m⁻²*h⁻¹). This resulted in a calculated loading flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected until the OD280 value dropped below 0.5 AU (1 cm UV cell). Thereafter the collecting was stopped. Thereby a final filtration pool (comprising the filter flowthrough and the flushing flowthrough) was obtained.
5) Thereafter, the alkaline regeneration solution was applied to the filter with the same flow rate as the intermediated “Affinity Pool pH 5.5” (7.67 mL/min (200 LMH)). Thus, the contact time was approx. 30 minutes.
6) To remove the regeneration solution from the filter, a water wash with same conditions as described in “filter conditioning: 1)” was carried out. In total, the filter was at a pH value above 10 for approximately 50 minutes.
7) To equilibrate the filter for the next filtration cycle, equilibration buffer was applied with same conditions as described in “filter conditioning: 2)”.
8) Steps 1) to 7) were repeated for further nine product filtration cycles (steps 1) to 4)) and further eight regeneration cycles (steps 5) to 7)). In total, 6 kg/m² (10×0.6 kg/m²) antibody were applied to the filter.
9) The separate final filtration pools were kept for analysis at −80° C.

Analyses and Results:

The following analyses were performed with the respective final filtration pools:
protein concentration (using the absorbance of 1.43 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)
MCE (Caliper)

The results are shown in the following Table X-1:

| | Final | | | | SEC | SEC | | |
|---|---|---|---|---|---|---|---|---|
| | Filtrat | | Photo- | | Area- | [LMW | | |
| | Vol. | Yield | Conc. | SEC | [MainPeak | Area- | DNA | cobas_HCP |
| Filtration | [mL] | [%] | [g/L] | [HMW [symbol] | Area-%] | %] | [ppb] | [ppm] |
| Load | not | not | 11.57 | 7.6 | 80.0 | 12.4 | 2212.62 | 15417 |
| X0SP | applicable | applicable | | | | | | |
| #1 | 334.35 | 81.06 | 3.51 | 4.4 | 92.6 | 3.0 | 6.05 | 1111 |
| w/o Reg. | | | | | | | | |
| #2 | 331.3 | 76.57 | 3.21 | 4.6 | 94.6 | 0.8 | 0.25 | 196 |
| #3 | 329.2 | 68.28 | 2.88 | 4.6 | 94.3 | 1.1 | 2.78 | 162 |
| #4 | 298.51 | 50.92 | 2.37 | 3.6 | 83.0 | 13.3 | 3.38 | 132 |
| #5 | 323.74 | 72.87 | 3.13 | 2.9 | 83.9 | 13.3 | 9.22 | 207 |
| #6 | 330.0 | 84.21 | 3.54 | 3.5 | 83.7 | 12.8 | 7.62 | 496 |
| #7 | 331.28 | 87.27 | 3.66 | 3.8 | 83.3 | 12.9 | 7.98 | 881 |
| #8 | 331.65 | 89.17 | 3.73 | 3.8 | 83.3 | 12.9 | 10.34 | 1117 |
| #9 | 331.51 | 88.68 | 3.71 | 3.8 | 83.3 | 12.9 | 3.42 | 1352 |
| #10 | 331.33 | 89.06 | 3.73 | 3.9 | 83.2 | 12.9 | 10.99 | 1532 |

| | | | | LEAP | |
|---|---|---|---|---|---|
| | MCE | MCE | | Average | LEAP |
| | [HMW | [MainPeak | MCE | Converted Rate | [hydrolytic |
| Filtration | %-CPA] | %-CPA] | [LMW % CPA] | [μM MU/h] | activity %] |
| Load | 1.0 | 69.5 | 29.5 | 7.9 | 100.0% |
| X0SP | | | | | |
| #1 | 1.1 | 81.0 | 17.9 | 2.3 | 29.1% |
| w/o Reg. | | | | | |
| #2 | 1.1 | 86.4 | 12.6 | 1.2 | 15.2% |
| #3 | 1.6 | 86.8 | 11.7 | 1.0 | 12.7% |
| #4 | 0.9 | 72.8 | 26.2 | 1.4 | 17.7% |
| #5 | 0.5 | 73.0 | 26.5 | N/A | N/A |
| #6 | 0.6 | 71.4 | 28.0 | 2.2 | 27.8% |
| #7 | 0.8 | 70.3 | 28.9 | 2.2 | 27.8% |
| #8 | 1.0 | 70.8 | 28.3 | 2.5 | 31.6% |
| #9 | 0.9 | 69.8 | 29.2 | N/A | N/A |
| #10 | 1.0 | 69.7 | 29.2 | 2.4 | 30.4% |

| Filtration | Mass Mainpeak [mg] | Yield Mainpeak [%] |
|---|---|---|
| Load X0SP | 1157.30 | 100.0% |
| #1 | 1086.67 | 93.9% |
| w/o Reg. | | |
| #2 | 1006.48 | 87.0% |
| #3 | 893.62 | 77.2% |
| #4 | 587.50 | 50.8% |
| #5 | 849.69 | 73.4% |
| #6 | 977.28 | 84.4% |
| #7 | 1010.42 | 87.3% |
| #8 | 1030.79 | 89.1% |
| #9 | 1024.26 | 88.5% |
| #10 | 1028.16 | 88.8% |

N/A = not analyzed [symbol]
indicates data missing or illegible when filed

### Example 2

Filtration of a Bispecific Anti-CD20/TfR Antibody Solution with a Silica-Containing Millipore Millistak+® HC Pro X0SP Filter, Regenerated with Alkaline Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Millipore Millistak+® HC Pro X0SP filter with 23 cm² filter area; Lot.: CP0BB08624
3) equilibration buffer: 40 mM sodium acetate, adjusted to pH 5.5 in purified water type II (Millipore Super Q)
4) alkaline regeneration solution: 1 M NaCl, 0.5 M NaOH, pH 12.6

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

Experimental Setup:

Following steps were performed sequentially:
1) An intermediate “Affinity Pool pH 5.5” containing the bispecific anti-CD20/TfR antibody was applied to the conditioned 23 cm² X0SP filter unit. The mass throughput was 800 g/m². The corresponding calculated volume throughput was 100 L/m². The feed flow was adjusted to 200 LMH (liter per square meter per hour; L*m⁻²*h⁻¹). This resulted in a calculated loading flow of 7.67 mL/min. The maximum feed pressure was 5.0 bar.
2) The filter flowthrough was not collected until the OD280 (280 nm) exceeded 0.5 AU determined using a 1 cm light path length UV-cell]. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected until OD280 was above 0.5 AU [1 cm UV cell]. When the OD280 value dropped below 0.5 AU (1 cm UV cell), the collecting was stopped. Thereby a final filtration pool (comprising the filter flowthrough and the flushing flowthrough) was obtained.
5) Thereafter, the alkaline regeneration solution was applied to the filter with the same flow rate as the intermediated “Affinity Pool pH 5.5” (7.67 mL/min (200 LMH)). Thus, the contact time was approx. 30 minutes.
6) To remove the regeneration solution from the filter, a water wash with same conditions as described in “filter conditioning: 1)” was carried out. In total, the filter was at a pH value above 10 for approximately 50 minutes.
7) To equilibrate the filter for the next filtration cycle, equilibration buffer was applied with same conditions as described in “filter conditioning: 2)”.
8) Steps 1) to 7) were repeated for further four product filtration cycles (steps 1) to 4)) and further three regeneration cycles (steps 5) to 7)). In total, 4 kg/m² (5×0.8 kg/m²) antibody were applied to the filter.
9) The separate final filtration pools were kept for analysis at −80° C.

Analyses and Results:

The following analyses were done:
protein concentration (using the absorbance of 1.57 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-2:

| | Final | | | | | |
|---|---|---|---|---|---|---|
| | Filtrat | | Photo- | SEC | SEC | SEC |
| | Vol. | Yield | Conc. | [HMW | [MainPeak | [LMW |
| Filtration | [mL] | [%] | [g/L] | Area-% ] | Area-% ] | Area-%] |
| Load | not | not | 8.07 | 13.9 | 81.0 | 5.11 |
| X0SP | applicable | applicable | | | | |
| #1 | 295.83 | 87.69 | 5.45 | 6.3 | 89.2 | 4.5 |
| w/o Reg. | | | | | | |
| #2 | 281.21 | 78.69 | 5.15 | 6.6 | 89.8 | 3.6 |
| #3 | 429.12 | 82.05 | 3.52 | 5.0 | 90.7 | 4.3 |
| #4 | 458.05 | 92.21 | 3.70 | 7.0 | 87.3 | 5.7 |
| #5 | 449.38 | 92.91 | 3.80 | 7.4 | 86.7 | 6.0 |
| #6 | 442.34 | 93.73 | 3.90 | 7.5 | 86.6 | 5.9 |
| #7 | 440.08 | 94.02 | 3.93 | 7.6 | 86.5 | 5.9 |
| #8 | 437.41 | 94.09 | 3.96 | 7.5 | 86.2 | 6.4 |

| | LEAP | | | |
|---|---|---|---|---|
| | Average | | | |
| | Converted | LEAP | | |
| | Rate [μM | [hydrolytic | | |
| Filtration | MU/h] | activity %] | DNA [ppb] | cobas_HCP [ppm] |
| Load X0SP | 15.6 | 100.0% | 109.54 | 18388 |
| #1 | 10.6 | 67.9% | 1.93 | 849 |
| w/o Reg. | | | | |
| #2 | 5.9 | 37.8% | 1.55 | 645 |
| #3 | 11.1 | 71.2% | 2.27 | 1384 |
| #4 | 12.8 | 82.1% | 3.10 | 3261 |
| #5 | 13.0 | 83.3% | 4.70 | 3978 |
| #6 | 12.4 | 79.5% | 5.59 | 4417 |
| #7 | 12.7 | 81.4% | 5.80 | 4718 |
| #8 | 12.7 | 81.4% | 5.36 | 4750 |

| Filtration | Mass Mainpeak [mg] | Yield Mainpeak [%] |
|---|---|---|
| Load X0SP | 1517.5 | 100.0% |
| #1 | 1438.8 | 94.8% |
| w/o Reg. | | |
| #2 | 1300.3 | 85.7% |
| #3 | 1369.5 | 90.2% |
| #4 | 1480.3 | 97.5% |
| #5 | 1480.4 | 97.6% |
| #6 | 1494.3 | 98.5% |
| #7 | 1495.3 | 98.5% |
| #8 | 1493.0 | 98.4% |

### Example 3

Filtration of a Bispecific Anti-CD20/TfR Antibody Solution with a Silica-Containing Millipore Millistak+® HC Pro X0SP Filter, Regenerated with an Acidic Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Millipore Millistak+® HC Pro X0SP filter with 23 cm² filter area; Lot.: CP0BB08624
3) equilibration buffer: 40 mM sodium acetate, adjusted to pH 5.5 in purified water type II (Millipore Super Q)
4) acidic regeneration solution: 167 mM acidic acid, 300 mM phosphoric acid, pH 1.34.

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

Experimental Setup:

Following steps were performed sequentially:
1) An intermediate “Affinity Pool pH 5.5” containing the bispecific anti-CD20/TfR antibody was applied to the conditioned 23 cm² X0SP filter unit. The mass throughput was 814 g/m². The corresponding calculated volume throughput was 100 L/m². The feed flow was adjusted to 200 LMH (liter per square meter per hour; L*m⁻²*h⁻¹). This resulted in a calculated loading flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected until the OD280 value dropped below 0.5 AU (1 cm UV cell). Thereafter the collecting was stopped. Thereby a final filtration pool (comprising the filter flowthrough and the flushing flowthrough) was obtained.
5) Thereafter, the acidic regeneration solution was applied to the filter with the same flow rate as the intermediated “Affinity Pool pH 5.5” (7.67 mL/min (200 LMH)). Thus, the contact time was approx. 30 minutes.
6) To remove the regeneration solution from the filter, a water wash with same conditions as described in “filter conditioning: 1)” was carried out. In total, the filter was at a pH value below 2 for approximately 50 minutes.
7) To equilibrate the filter for the next filtration cycle, equilibration buffer was applied with same conditions as described in “filter conditioning: 2)”.
8) Steps 1) to 7) were repeated for further four product filtration cycles (steps 1) to 4)) and further three regeneration cycles (steps 5) to 7)). In total, 4.07 kg/m² (5×0.814 kg/m²) antibody were applied to the filter.
9) The separate final filtration pools were kept for analysis at −80° C.

Analyses and Results:

The following analyses were performed with the respective final filtration pools:
protein concentration (using the absorbance of 1.57 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-3:

| | Final | | | | | |
|---|---|---|---|---|---|---|
| | Filtrat | | Photo- | SEC | SEC | SEC |
| | Vol. | Yield | Conc. | [HMW | [MainPeak | [LMW |
| Filtration | [mL] | [%] | [g/L] | Area-%] | Area-% ] | Area-%] |
| Load X0SP | not | not | 8.22 | 0.67 | 79.87 | 4.79 |
| | applicable | applicable | | | | |
| #1 w/o Reg. | 310.23 | 85.5 | 5.16 | 0.54 | 88.794 | 3.96 |
| #2 | 314.92 | 87.36 | 5.2 | 0.57 | 88.491 | 4.04 |
| #3 | 307.17 | 87.07 | 5.31 | 0.57 | 88.235 | 4.12 |
| #4 | 306.86 | 88.12 | 5.38 | 0.57 | 88.11 | 4.09 |
| #5 | 310.83 | 88.19 | 5.31 | 0.61 | 87.838 | 4.16 |

| | LEAP | | | |
|---|---|---|---|---|
| | Average | | | |
| | Converted | LEAP | | |
| | Rate [μM | [hydrolytic | | |
| Filtration | MU/h] | activity %] | DNA [ppb] | cobas_HCP [ppm] |
| Load X0SP | 15.60 | 100.0% | 276.31 | 17340 |
| #1 w/o Reg. | 10.00 | 64.1% | 1.55 | 992 |
| #2 | 10.20 | 65.4% | 1.68 | 895 |
| #3 | 10.20 | 65.4% | 2.21 | 930 |
| #4 | 10.60 | 67.9% | 2.04 | 1043 |
| #5 | 10.70 | 68.6% | 2.27 | 1115 |

| Filtration | Mass Mainpeak [mg] | Yield Mainpeak [%] |
|---|---|---|
| Load X0SP | 1497.00 | 100% |
| #1 w/o Reg. | 1421.40 | 95.0% |
| #2 | 1449.11 | 96.8% |
| #3 | 1439.18 | 96.1% |
| #4 | 1454.61 | 97.2% |
| #5 | 1449.77 | 96.8% |

### Example 4

Filtration of an IgG Antibody IL-2 Fusion Protein Solution with a Silica-Containing Millipore Millistak+® HC Pro X0SP Filter, Regenerated with an Acidic Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Millipore Millistak+® HC Pro X0SP filter with 23 cm² filter area; Lot.: CP0BB08624
3) equilibration buffer: 150 mM acidic acid/tris, adjusted to pH 5.5 in purified water type II (Millipore Super Q)
4) acidic regeneration solution: 167 mM acidic acid, 300 mM phosphoric acid, pH 1.34.

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing the IgG-IL2 antibody fusion protein was applied to the conditioned 23 cm² X0SP filter unit. The mass throughput was 548 g/m². The corresponding calculated volume throughput was 56 L/m². The feed flow was adjusted to 200 LMH (liter per square meter per hour; L*m⁻²*h⁻¹). This resulted in a calculated loading flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected until the OD280 value dropped below 0.5 AU (1 cm UV cell). Thereafter the collecting was stopped. Thereby a final filtration pool (comprising the filter flowthrough and the flushing flowthrough) was obtained.
5) Thereafter, the acidic regeneration solution was applied to the filter with the same flow rate as the intermediated “Affinity Pool pH 5.5” (7.67 mL/min (200 LMH)). Thus, the contact time was approx. 30 minutes.
6) To remove the regeneration solution from the filter, a water wash with same conditions as described in “filter conditioning: 1)” was carried out. In total, the filter was at a pH value below 2 for approximately 50 minutes.
7) To equilibrate the filter for the next filtration cycle, equilibration buffer was applied with same conditions as described in “filter conditioning: 2)”.
8) Steps 1) to 7) were repeated for further five product filtration cycles (steps 1) to 4)) and further four regeneration cycles (steps 5) to 7)). In total, 2.288 kg/m² (6×0.548 kg/m²) fusion protein was applied to the filter.
9) The separate final filtration pools were kept for analysis at −80° C.

Analyses and Results:

The following analyses were performed with the respective final filtration pools:
protein concentration (using the absorbance of 1.25 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-4:

| | Final | | Photo- | SEC | SEC | SEC |
|---|---|---|---|---|---|---|
| | Filtrat | Yield | Conc. | [HMW | [MainPeak | [LMW |
| Filtration | Vol. [mL] | [%] | [g/L] | Area-%] | Area-% ] | Area-%] |
| Load | not | not | 9.86 | 6.64% | 93.30% | 0.05% |
| X0SP | applicable | applicable | | | | |
| #1 w/o Reg. | 204.76 | 81.48% | 5.02 | 2.38% | 97.60% | 0.02% |
| #2 | 212.09 | 86.59% | 5.15 | 2.52% | 97.43% | 0.05% |
| #3 | 214.61 | 87.12% | 5.12 | 2.63% | 97.33% | 0.04% |
| #4 | 210.51 | 87.74% | 5.25 | 2.78% | 97.16% | 0.06% |
| #5 | 211.18 | 87.74% | 5.24 | 2.95% | 97.00% | 0.04% |
| #6 | 216.11 | 88.47% | 5.17 | 2.82% | 97.15% | 0.03% |

| | LEAP | | | |
|---|---|---|---|---|
| | Average | | | |
| | Converted | LEAP | | |
| | Rate [μM | [hydrolytic | | |
| Filtration | MU/h] | activity %] | DNA [ppb] | cobas_HCP [ppm] |
| Load X0SP | 9.1 | 100% | 1428 | 7315 |
| #1 w/o Reg. | 1.9 | 21% | 1.59 | 17 |
| #2 | 2.1 | 23% | 1.55 | 20 |
| #3 | 2.3 | 25% | 1.56 | 23 |
| #4 | 2.1 | 23% | 1.52 | 24 |
| #5 | 2.0 | 22% | 1.53 | 31 |
| #6 | 2.3 | 25% | 1.55 | 33 |

| Filtration | Mass Mainpeak [mg] | Yield Mainpeak [%] |
|---|---|---|
| Load X0SP | 1,287.91 | 100% |
| #1 w/o Reg. | 1,003.23 | 78% |
| #2 | 1,064.19 | 83% |
| #3 | 1,069.47 | 83% |
| #4 | 1,073.79 | 83% |
| #5 | 1,073.39 | 83% |
| #6 | 1,085.45 | 84% |

### Example 5

Filtration of an IgG-IL2 Fusion Polypeptide Solution with a Silica-Containing Pall PDD1 SUPRAcap 50 Filter, Regenerated with an Acidic Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Pall PDD1 SUPRAcap 50 (SC050PDD1) filter with 22 cm² filter area; Lot.: 103864042
3) equilibration buffer: 150 mM acetic acid/Tris, adjusted to pH 5.5 in purified water type II (Millipore Super Q)
4) acidic regeneration solution: 167 mM acetic acid, 300 mM phosphoric acid, pH 1.34

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing the IgG-IL2 fusion polypeptide was applied to the conditioned 22 cm² PDD1 filter unit. The mass throughput was 547 g/m². The corresponding calculated volume throughput was 55 L/m². The feed flow was adjusted to 191 LMH (liter per square meter per hour; L*m⁻²*h⁻¹). This resulted in a calculated loading flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected until the OD280 value dropped below 0.5 AU (1 cm UV cell). Thereafter the collecting was stopped. Thereby a final filtration pool (comprising the filter flowthrough and the flushing flowthrough) was obtained.
5) Thereafter, the acidic regeneration solution was applied to the filter with the same flow rate as the intermediated “Affinity Pool pH 5.5” (7.67 mL/min (191 LMH)). Thus, the contact time was approx. 30 minutes.
6) To remove the regeneration solution from the filter, a water wash with same conditions as described in “filter conditioning: 1)” was carried out. In total, the filter was at a pH value below 2 for approximately 50 minutes.
7) To equilibrate the filter for the next filtration cycle, equilibration buffer was applied with same conditions as described in “filter conditioning: 2)”.
8) Steps 1) to 7) were repeated for further five product filtration cycles (steps 1) to 4)) and further four regeneration cycles (steps 5) to 7)). In total, 3.282 kg/m² (6×0.547 kg/m²) fusion protein was applied to the filter.
9) The separate final filtration pools were kept for analysis at −80° C.

Analyses and Results:

The following analyses were performed with the respective final filtration pools:
protein concentration (using the absorbance of 1.25 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-5:

| | Final | | | |
|---|---|---|---|---|
| | Filtrat | | Photo- | |
| | Vol. | Yield | Conc. | |
| Filtration | [mL] | [%] | [g/L] | |
| Load | not | not | 9.86 | |
| PDD1 | applicable | applicable | | |
| #1 w/o | 164.28 | 83.73 | 6.13 | |
| Reg. | | | | |
| #2 | 155.72 | 90.3 | 6.97 | |
| #3 | 154.46 | 92.15 | 7.17 | |
| #4 | 155.12 | 91.91 | 7.12 | |
| #5 | 156 | 92.28 | 7.11 | |
| #6 | 154.6 | 91.85 | 7.14 | |
| | LEAP | | | |
| | Average | | | |
| | Converted | LEAP | | |
| | Rate [μM | [hydrolytic | | cobas_HCP |
| Filtration | MU/h] | activity %] | DNA [ppb] | [ppm] |
| Load PDD1 | 9.1 | 100% | 1428 | 7315 |
| #1 w/o Reg. | 4.7 | 52% | 310.00 | 1299 |
| #2 | 3.4 | 37% | 58.22 | 816 |
| #3 | 3.4 | 37% | 69.97 | 898 |
| #4 | 3.7 | 41% | 81.68 | 962 |
| #5 | 3.9 | 43% | 94.75 | 1055 |
| #6 | 3.9 | 43% | 138.85 | 1106 |

### Example 6

Filtration of Crovalimab (Anti-C5 Antibody) Solution with a Silica-Containing Millipore Millistak+® HC Pro X0SP Filter Unit, with Water and Buffer Application (without Alkaline or Acidic Filter Regeneration)—Comparative Example

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Millipore Millistak+® HC Pro X0SP filter with 23 cm² filter area; Lot.: CP0BB08624
3) equilibration buffer: 150 mM acetic acid/Tris, adjusted to pH 5.5 in purified water type II (Millipore Super Q)
4) intermediate solution: water and equilibration buffer

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing crovalimab was applied to the conditioned 23 cm² X0SP filter unit. The mass throughput was 600 g/m². The corresponding calculated volume throughput was 41.3 L/m². The feed flow was adjusted to 200 LMH (liter per square meter per hour; L*m⁻²*h⁻¹). This resulted in a calculated loading flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flowthrough of the filter was collected for 70 L/m². When the flowthrough of 70 L/m² was reached, the collecting was stopped. Thereby a final filtration pool was obtained.
5) To prepare the filter for the next filtration cycle, a water wash with same conditions as described in “filter conditioning: 1)” was carried out.
6) To equilibrate the filter for the next filtration cycle, equilibration buffer was applied with same conditions as described in “filter conditioning: 2)”.
7) Steps 1) to 6) were repeated for further nine product filtration cycles without harsh regeneration solution between the filtration cycles. In total, 6.0 kg/m² (10×0.6 kg/m²) antibody were applied to the filter.
8) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out ofloaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)
MCE (Caliper)

The results are shown in the following Table X-6:

| | Final | | | | | |
|---|---|---|---|---|---|---|
| | Filtration | | | | | |
| | Pool | | Photo- | SEC | SEC | SEC |
| | Vol. | Yield | Conc. | [HMW | [MainPeak | [LMW |
| Filtration | [mL] | [%] | [g/L] | Area-%] | Area-% ] | Area-%] |
| Load | not | not | 14.53 | 5.35 | 93.66 | 0.99 |
| X0SP | applicable | applicable | | | | |
| #1 | 235.13 | 87.15% | 5.12 | 1.22 | 98.11 | 0.67 |
| w/o Reg. | | | | | | |
| #2 | 237.54 | 90.23% | 5.24 | 1.45 | 97.80 | 0.76 |
| #3 | 238.78 | 90.68% | 5.24 | 1.39 | 97.85 | 0.75 |
| #4 | 239.49 | 93.07% | 5.36 | 1.67 | 97.58 | 0.75 |
| #5 | 240.01 | 92.98% | 5.35 | 2.27 | 95.80 | 0.94 |
| #6 | 240.01 | 93.72% | 5.39 | 3.78 | 95.28 | 0.94 |
| #7 | 240.64 | 94.68% | 5.43 | 4.03 | 95.01 | 0.96 |
| #8 | 240.84 | 95.10% | 5.45 | 4.20 | 94.84 | 0.96 |
| #9 | 241.44 | 95.12% | 5.44 | 4.29 | 94.87 | 0.84 |
| #10 | 241.64 | 95.53% | 5.46 | 4.42 | 94.61 | 0.98 |

| | LEAP | | | |
|---|---|---|---|---|
| | Average | | | |
| | Converted | LEAP | | |
| | Rate [μM | [hydrolytic | | |
| Filtration | MU/h] | activity %] | DNA [ppb] | cobas_HCP [ppm] |
| Load X0SP | 20.2 | 100% | 8176.19 | 12529 |
| #1 w/o Reg. | 1.1 | 5% | 0.16 | 2 |
| #2 | 1.5 | 7% | 0.15 | 306 |
| #3 | N/A | N/A | 0.15 | 1243 |
| #4 | 3.2 | 16% | 1.49 | 2913 |
| #5 | N/A | N/A | 12.83 | 4042 |
| #6 | 8.2 | 41% | 315.39 | 4815 |
| #7 | 10.1 | 50% | 1185.76 | 4998 |
| #8 | 10.3 | 51% | 1654.83 | 5347 |
| #9 | 11 | 54% | 2070.50 | 5826 |
| #10 | 12 | 59% | 2349.28 | 2006 |

| Filtration | Mass Mainpeak [mg] | Yield Mainpeak [%] |
|---|---|---|
| Load X0SP | 1292.84 | 100% |
| #1 w/o Reg. | 1180.21 | 91% |
| #2 | 1218.14 | 94% |
| #3 | 1224.73 | 95% |
| #4 | 1253.61 | 97% |
| #5 | 1229.57 | 95% |
| #6 | 1232.62 | 95% |
| #7 | 1241.72 | 96% |
| #8 | 1245.01 | 96% |
| #9 | 1245.67 | 96% |
| #10 | 1247.56 | 96% |

N/A = not analyzed

### Example 7

Filtration of Crovalimab (Anti-C5 Antibody) Solution with a Silica-Containing Millipore Millistak+® HC Pro X0SP Filter, Derivatized with an Alkaline Pre-Treatment, Regenerated with an Alkaline Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Millipore Millistak+® HC Pro X0SP filter with 23 cm² filter area; Lot.: CP0BB08624
3) equilibration buffer: 150 mM acetic acid/tris, adjusted to pH 5.5 in purified water type II (Millipore Super Q)
4) alkaline pre-treatment and regeneration solution: 1 M NaOH

Filter Derivatization:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.
3) 100 L/m² alkaline pre-treatment/regeneration solution were applied to/flown through the filter. The system flow was paused after 70 L/m² flowthrough for four hours to incubate the filter with the alkaline regeneration solution. The regeneration flow was 7.67 mL/min at a max. feed pressure of 5.0 bar.

### Experimental Setup

Following steps were performed sequentially:
1) An intermediate “Affinity Pool pH 5.5” containing crovalimab was applied to the derivatized 23 cm² X0SP filter unit. The mass throughput was 600 g/m². The corresponding calculated volume throughput was 41.3 L/m². The feed flow was adjusted to 200 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flowthrough of the filter was collected for 70 L/m². When the flowthrough of 70 L/m² was reached, the collecting was stopped. Thereby a final filtration pool was obtained.
5) Thereafter, the alkaline regeneration solution was applied to the filter with the same flow rate as the intermediated “Affinity Pool pH 5.5” (7.67 mL/min (200 LMH)). Thus, the contact time was approx. 30 minutes.
6) To remove the regeneration solution from the filter, a water wash with same conditions as described in “filter derivatization: 1)” was carried out. In total the filter was at a pH value above 10 for approximately 50 minutes.
7) To equilibrate the filter for the next filtration cycle, equilibration buffer was applied with same conditions as described in “filter derivatization: 2)”.
8) Steps 1) to 7) were repeated for further nine product filtration cycles (steps 1) to 4)) and further eight regeneration cycles (steps 5) to 7)). In total, 6.0 kg/m² (10×0.6 kg/m²) antibody were applied to the filter.
9) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-7:

| | Final | | | | | |
|---|---|---|---|---|---|---|
| | Filtration | | Photo- | SEC | SEC | SEC |
| | Pool Vol. | Yield | Conc. | [HMW | [MainPeak | [LMW |
| Filtration | [mL] | [%] | [g/L] | Area-% ] | Area-% ] | Area-%] |
| Load | not | not | 14.53 | 3.5 | 94.9 | 1.6 |
| X0SP | applicable | applicable | | | | |
| #1 | 233.38 | 87.36% | 5.17 | 0.3 | 98.5 | 1.2 |
| #2 | 234.8 | 89.71% | 5.27 | 0.6 | 98 | 1.3 |
| #3 | 236.19 | 91.27% | 5.33 | 0.7 | 98 | 1.4 |
| #4 | 233.36 | 92.03% | 5.44 | 0.7 | 97.9 | 1.4 |
| #5 | 230.28 | 92.36% | 5.53 | 0.7 | 97.9 | 1.3 |
| #6 | 231.01 | 92.23% | 5.51 | 0.8 | 97.8 | 1.4 |
| #7 | 231.09 | 92.32% | 5.51 | 0.8 | 97.9 | 1.4 |
| #8 | 230.78 | 92.60% | 5.54 | 0.8 | 97.8 | 1.4 |
| #9 | 230.61 | 93.11% | 5.57 | 0.9 | 97.7 | 1.4 |
| #10 | 230.65 | 92.75% | 5.55 | 0.8 | 97.8 | 1.4 |

| | LEAP | | | |
|---|---|---|---|---|
| | Average | | | |
| | Converted | LEAP | | |
| | Rate [μM | [hydrolytic | | |
| Filtration | MU/h] | activity %] | DNA [ppb] | cobas_HCP [ppm] |
| Load X0SP | 13.7 | 100% | 8176.1 | 12529 |
| #1. | 3.3 | 24% | 18.0 | 848 |
| #2 | 3.3 | 24% | 398.2 | 1920 |
| #3 | 3.7 | 27% | 364.4 | 2315 |
| #4 | N/A | N/A | 378.4 | 2460 |
| #5 | 3.9 | 28% | 379.3 | 2682 |
| #6 | 4.4 | 32% | 391.9 | 2746 |
| #7 | 4.7 | 34% | 402.6 | 2744 |
| #8 | 4.5 | 33% | 436.9 | 3033 |
| #9 | 4.4 | 32% | 412.7 | 3072 |
| #10 | 4.6 | 34% | 396.3 | 3064 |

| Filtration | Mass Mainpeak [mg] | Yield Mainpeak [%] |
|---|---|---|
| Load X0SP | 1309.95 | 100% |
| #1. | 1187.74 | 91% |
| #2 | 1213.57 | 93% |
| #3 | 1234.71 | 94% |
| #4 | 1243.69 | 95% |
| #5 | 1247.52 | 95% |
| #6 | 1245.09 | 95% |
| #7 | 1247.56 | 95% |
| #8 | 1250.10 | 95% |
| #9 | 1255.74 | 96% |
| #10 | 1252.17 | 96% |

N/A = not analyzed

### Example 8

Filtration of Crovalimab (Anti-C5 Antibody) Solution with a Silica-Containing Millipore Millistak+® HC Pro X0SP Filter, Regenerated with an Acidic Treatment

Materials:
1) Experiments were performed with an Äkta Avant 150 (Cytiva, Uppsala, Sweden) chromatographic skid. The filter was installed to the column valve instead of a column, Pressure, pH value, conductivity, OD280 were monitored. The applied volume was regulated by a sample pump.
2) Millipore Millistak+® HC Pro X0SP filter with 23 cm² filter area; Lot.: CP0BB08624
3) equilibration buffer: 150 mM acetic acid/tris, adjusted to pH 5.5 in purified water type II (Millipore Super Q)
4) acid regeneration solution: 0.5 M phosphoric acid

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

Experimental Setup:

Following steps were performed sequentially:
1) An intermediate “Affinity Pool pH 5.5” containing crovalimab was applied to the conditioned 23 cm² X0SP filter unit. The mass throughput was 600 g/m². The corresponding calculated volume throughput was 41.3 L/m². The feed flow was adjusted to 200 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flowthrough of the filter was collected for 70 L/m². When the flowthrough of 70 L/m² was reached, the collecting was stopped. Thereby a final filtration pool was obtained.
5) Thereafter, the acidic regeneration solution was applied to the filter with the same flow rate as the intermediated “Affinity Pool pH 5.5” (7.67 mL/min (200 LMH)). Thus, the contact time was approx. 30 minutes.
6) To remove the regeneration solution from the filter, a water wash with same conditions as described in “filter conditioning: 1)” was carried out. In total, the filter was at a pH value below 2 for approximately 50 minutes.
7) To equilibrate the filter for the next filtration cycle, equilibration buffer was applied with same conditions as described in “filter conditioning: 2)”.
8) Steps 1) to 7) were repeated for further nine product filtration cycles (steps 1) to 4)) and further eight regeneration cycles (steps 5) to 7)). In total, 6.0 kg/m² (10×0.6 kg/m²) antibody were applied to the filter.
9) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-8:

| | Final | | | | | |
|---|---|---|---|---|---|---|
| | Filtration | | Photo- | SEC | SEC | SEC |
| | Pool Vol. | Yield | Conc. | [HMW | [MainPeak | [LMW |
| Filtration | [mL] | [%] | [g/L] | Area-%] | Area-%] | Area-%] |
| Load X0SP | not | not | 14.53 | 3.5 | 94.9 | 1.6 |
| | applicable | applicable | | | | |
| #1 w/o Reg. | 223.14 | 84.06% | 5.20 | 0.1 | 98.9 | 1.1 |
| #2 | 224.71 | 86.12% | 5.29 | 0.2 | 98.7 | 1.2 |
| #3 | 225.46 | 85.63% | 5.24 | 0.2 | 98.7 | 1.2 |
| #4 | 225.83 | 85.84% | 5.25 | 0.2 | 98.6 | 1.2 |
| #5 | 225.99 | 86.24% | 5.27 | 0.2 | 98.7 | 1.2 |
| #6 | 226.25 | 86.92% | 5.30 | 0.2 | 98.6 | 1.2 |
| #7 | 226.54 | 86.82% | 5.29 | 0.2 | 98.6 | 1.2 |
| #8 | 226.67 | 86.64% | 5.28 | 0.2 | 98.5 | 1.3 |
| #9 | 226.84 | 86.80% | 5.28 | 0.2 | 98.5 | 1.2 |
| #10 | 227.28 | 87.44% | 5.31 | 0.3 | 98.5 | 1.3 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] |
| Load X0SP | 13.7 | 100% | 8176.19 | 12529 | 1309.95 | 100% |
| #1 w/o Reg. | 1.2 | 9% | 0.15 | 12 | 1147.56 | 88% |
| #2 | 1.5 | 11% | 0.15 | 44 | 1174.28 | 90% |
| #3 | 1.5 | 11% | 0.15 | 54 | 1166.59 | 89% |
| #4 | 1.2 | 9% | 0.15 | 64 | 1168.36 | 89% |
| #5 | 1.4 | 10% | 1.52 | 74 | 1174.90 | 90% |
| #6 | 1.5 | 11% | 1.51 | 74 | 1183.03 | 90% |
| #7 | 1.7 | 12% | 1.51 | 83 | 1181.69 | 90% |
| #8 | 1.8 | 13% | 1.52 | 93 | 1177.95 | 90% |
| #9 | 1.7 | 12% | 1.51 | 105 | 1180.11 | 90% |
| #10 | 1.4 | 10% | 1.51 | 101 | 1188.93 | 91% |

N/A = not analyzed

### Example 9

Filtration of a Crovalimab (Anti-C5 Antibody) Solution with a Silica-Containing Pall PDD1 SUPRAcap 50 Filter, with Water and Buffer Application (without Alkaline or Acidic Filter Regeneration)—Comparative Example

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Pall PDD1 SUPRAcap 50 (SC050PDD1) filter with 22 cm² filter area; Lot.: 103864042
3) equilibration buffer: 150 mM acetic acid/tris, adjusted to pH 5.5 in purified water type II (Millipore Super Q)
4) intermediate solution: water and equilibration buffer

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing crovalimab was applied to the conditioned 22 cm² PDD1 filter unit. The mass throughput was 599 g/m². The corresponding calculated volume throughput was 120 L/m².

The feed flow was adjusted to 191 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flowthrough of the filter was collected for 70 L/m². When the flowthrough of 70 L/m² was reached, the collecting was stopped. Thereby a final filtration pool was obtained.
5) To prepare the filter for the next filtration cycle, a water wash with same conditions as described in “filter conditioning: 1)” was carried out.
6) To equilibrate the filter for the next filtration cycle, equilibration buffer was applied with same conditions as described in “filter conditioning: 2)”.
7) Steps 1) to 6) were repeated for further nine product filtration cycles without the application of harsh regeneration solution between the filtration cycles. In total, with 5.99 kg/m² (100×0.599 kg/m²) antibody were applied to the filter.
8) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-9:

| | Final | | | | | |
|---|---|---|---|---|---|---|
| | Filtration | | Photo- | SEC | SEC | SEC |
| | Pool Vol. | Yield | Conc. | [HMW | [MainPeak | [LMW |
| Filtration | [mL] | [%] | [g/L] | Area-%] | Area-%] | Area-%] |
| Load PDD1 | not | not | 4.97 | 3.5 | 94.9 | 1.6 |
| | applicable | applicable | | | | |
| #1 w/o Reg. | 397.73 | 88.08% | 2.92 | 0.1 | 98.9 | 1.0 |
| #2 | 408.22 | 92.89% | 3.00 | 0.4 | 98.3 | 1.3 |
| #3 | 408.18 | 94.64% | 3.05 | 1.1 | 97.5 | 1.4 |
| #4 | 409.48 | 94.98% | 3.06 | 1.4 | 97.2 | 1.4 |
| #5 | 409.49 | 96.60% | 3.11 | 1.8 | 96.7 | 1.4 |
| #6 | 409.78 | 96.76% | 3.11 | 2.5 | 95.9 | 1.5 |
| #7 | 410.01 | 96.78% | 3.11 | 2.6 | 95.9 | 1.5 |
| #8 | 410.05 | 97.23% | 3.12 | 2.7 | 95.7 | 1.6 |
| #9 | 410.26 | 97.65% | 3.13 | 2.5 | 96.0 | 1.5 |
| #10 | 410.39 | 97.74% | 3.14 | 2.2 | 96.3 | 1.5 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] |
| Load PDD1 | 16.2 | 100% | 5955.73 | 12114 | 1249.96 | 100% |
| #1 w/o Reg. | 1.0 | 6% | N/A | 27 | 1147.42 | 92% |
| #2 | 1.5 | 9% | 17.3 | 511 | 1202.64 | 96% |
| #3 | 5.1 | 31% | 753.1 | 2586 | 1215.42 | 97% |
| #4 | 8.9 | 55% | 2396.1 | 4036 | 1215.93 | 97% |
| #5 | 10.9 | 67% | 2825.9 | 4592 | 1230.30 | 98% |
| #6 | 11.8 | 73% | 3299.0 | 5495 | 1222.16 | 98% |
| #7 | 11.6 | 72% | 3859.8 | 5760 | 1222.46 | 98% |
| #8 | 12.4 | 77% | 4585.3 | 5961 | 1225.52 | 98% |
| #9 | 13.5 | 83% | 4357.3 | 6494 | 1230.78 | 98% |
| #10 | 12.5 | 77% | 4564.9 | 6487 | 1239.76 | 99% |

N/A = not analyzed

### Example 10

Filtration of a Crovalimab (Anti-C5 Antibody) Solution with a Silica-Containing Pall PDD1 SUPRAcap 50 Filter, Derivatized with an Alkaline Pre-Treatment, Regenerated with an Alkaline Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Pall PDD1 SUPRAcap 50 (SC050PDD1) filter with 22 cm² filter area; Lot.: 103864042
3) equilibration buffer: 150 mM acetic acid/Tris, adjusted to pH 5.5 in purified water type II (Millipore Super Q)
4) alkaline regeneration solution: 1 M NaOH

Filter Derivatization:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.
3) 100 L/m² alkaline pre-treatment/regeneration solution were applied to/flown through the filter. The system flow was paused after 70 L/m² flowthrough for four hours to incubate the filter with the alkaline regeneration solution. The regeneration flow was 7.67 mL/min at a max. feed pressure of 5.0 bar.

### Experimental Setup

Following steps were performed sequentially:
1) An intermediate “Affinity Pool pH 5.5” containing crovalimab was applied to the derivatized 22 cm² PDD1 filter unit. The mass throughput was 600 g/m². The corresponding calculated volume throughput was 41.3 L/m². The feed flow was adjusted to 191 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected for 70 L/m². When the flushing flowthrough of 70 L/m² was reached, the collecting was stopped. Thereby a final filtration pool (comprising the filter flowthrough and the flushing flowthrough) was obtained.
5) Thereafter, the alkaline regeneration solution was applied to the filter with the same flow rate as the intermediated “Affinity Pool pH 5.5” (7.67 mL/min (191 LMH)). Thus, the contact time was approx. 30 minutes.
6) To remove the regeneration solution from the filter, a water wash with same conditions as described in “filter derivatization: 1)” was carried out. In total the filter was at a pH value above 10 for approximately 50 minutes.
7) To equilibrate the filter for the next filtration cycle, equilibration buffer was applied with same conditions as described in “filter derivatization: 2)”.
8) Steps 1) to 7) were repeated for further nine product filtration cycles (steps 1) to 4)) and further eight regeneration cycles (steps 5) to 7)). In total, 6.0 kg/m² (10×0.6 kg/m²) antibody were applied to the filter.
9) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-10:

| | Final | | Photo- | SEC | SEC | SEC |
|---|---|---|---|---|---|---|
| | Filtrat | Yield | Conc. | [HMW | [MainPeak | [LMW |
| Filtration | Vol. [mL] | [%] | [g/L] | Area-%] | Area-%] | Area-%] |
| LoadP DD1 | not | not | 13.76 | 5.33 | 93.69 | 0.97 |
| | applicable | applicable | | | | |
| #1 | 228.18 | 82.43% | 4.772 | 1.3 | 98.07 | 0.63 |
| #2 | 227.38 | 81.16% | 4.715 | 1.25 | 98.16 | 0.59 |
| #3 | 227.1 | 80.51% | 4.683 | 1.26 | 98.14 | 0.59 |
| #4 | 226.94 | 80.18% | 4.667 | 1.27 | 98.13 | 0.59 |
| #5 | 226.32 | 79.39% | 4.634 | 1.27 | 98.14 | 0.59 |
| #6 | 225.79 | 79.41% | 4.646 | 1.27 | 98.13 | 0.6 |
| #7 | 225.4 | 80.20% | 4.7 | 1.29 | 98.12 | 0.6 |
| #8 | 227.46 | 79.85% | 4.637 | 1.42 | 97.97 | 0.61 |
| #9 | 240.44 | 80.33% | 4.413 | 1.25 | 98.19 | 0.57 |
| #10 | 244.99 | 80.16% | 4.3222 | 1.36 | 98.06 | 0.58 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] |
| Load PDD1 | 18.5 | 100% | 5595.93 | 10237 | 1237.61 | 100% |
| #1 | 1 | 5% | 0.17 | 23 | 1067.86 | 86% |
| #2 | 1.1 | 6% | 0.17 | 21 | 1052.37 | 85% |
| #3 | 1 | 5% | 0.17 | 23 | 1043.73 | 84% |
| #4 | 0.9 | 5% | 0.17 | 26 | 1039.32 | 84% |
| #5 | 0.9 | 5% | 1.73 | 25 | 1029.26 | 83% |
| #6 | 0.8 | 4% | 10.68 | 38 | 1029.40 | 83% |
| #7 | 1.1 | 6% | 122.13 | 204 | 1039.46 | 84% |
| #8 | 1.7 | 9% | 417.13 | 740 | 1033.32 | 83% |
| #9 | 2.6 | 14% | 802.18 | 1411 | 1041.86 | 84% |
| #10 | 3.6 | 19% | 1267.87 | 2265 | 1038.35 | 84% |

### Example 11

Filtration of a Crovalimab (Anti-C5 Antibody) Solution with a Silica-Containing Pall PDD1 SUPRAcap 50 Filter, Regenerated with an Acidic Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Pall PDD1 SUPRAcap 50 (SC050PDD1) filter with 22 cm² filter area; Lot.: 103864042
3) equilibration buffer: 150 mM acetic acid/Tris, adjusted to pH 5.5 in purified water type II (Millipore Super Q)
4) acid regeneration solution: 0.5 M phosphoric acid

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

Experimental Setup:

Following steps were performed sequentially:
1) An intermediate “Affinity Pool pH 5.5” containing crovalimab was applied to the conditioned 22 cm² PDD1 filter unit. The mass throughput was 600 g/m². The corresponding calculated volume throughput was 41.3 L/m². The feed flow was adjusted to 191 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected for 70 L/m². When the flushing flowthrough of 70 L/m² was reached, the collecting was stopped. Thereby a final filtration pool (comprising the filter flowthrough and the flushing flowthrough) was obtained.
5) Thereafter, the acidic regeneration solution was applied to the filter with the same flow rate as the intermediated “Affinity Pool pH 5.5” (7.67 mL/min (191 LMH)). Thus, the contact time was approx. 30 minutes.
6) To remove the regeneration solution from the filter, a water wash with same conditions as described in “filter conditioning: 1)” was carried out. In total the filter was at a pH value below 2 for approximately 50 minutes.
7) To equilibrate the filter for the next filtration cycle, equilibration buffer was applied with same conditions as described in “filter conditioning: 2)”.
8) Steps 1) to 7) were repeated for further nine product filtration cycles (steps 1) to 4)) and further eight regeneration cycles (steps 5) to 7)). In total, 6.0 kg/m² (10×0.6 kg/m²) antibody were applied to the filter.
9) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by aPRC_DNA
SE-HPLC (size exclusion HPLC)

The results are shown in the following Table X-11:

| | Final | | Photo- | SEC | SEC | SEC |
|---|---|---|---|---|---|---|
| | Filtrat | Yield | Conc. | [HMW | [MainPeak | [LMW |
| Filtration | Vol. [mL] | [%] | [g/L] | Area-%] | Area-%] | Area-%] |
| Load PDD1 | not | not | 13.76 | 4.2 | 94 | 1.8 |
| | applicable | applicable | | | | |
| #1 w/o Reg. | 229.84 | 87.17% | 4.96 | 0.1 | 98.8 | 1.1 |
| #2 | 233.03 | 90.84% | 5.10 | 0.2 | 98.5 | 1.3 |
| #3 | 233.22 | 91.06% | 5.10 | 0.2 | 98.4 | 1.3 |
| #4 | 233.56 | 90.98% | 5.09 | 0.3 | 98.4 | 1.3 |
| #5 | 233.82 | 91.32% | 5.11 | 0.2 | 98.5 | 1.3 |
| #6 | 234.01 | 90.80% | 5.07 | 0.3 | 98.3 | 1.3 |
| #7 | 234.1 | 90.84% | 5.07 | 0.3 | 98.4 | 1.3 |
| #8 | 234.12 | 90.83% | 5.07 | 0.4 | 98.3 | 1.3 |
| #9 | 234.16 | 90.77% | 5.07 | 0.4 | 98.3 | 1.3 |
| #10 | 234.23 | 91.29% | 5.09 | 0.4 | 98.2 | 1.4 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] |
| Load PDD1 | 18.5 | 100% | 5988.37 | 10742 | 1228.77 | 100% |
| #1 w/o Reg. | 1 | 5% | <0.16 | 43 | 1126.33 | 92% |
| #2 | 1.3 | 7% | <0.16 | 348 | 1170.63 | 95% |
| #3 | 1.5 | 8% | <0.16 | 420 | 1170.39 | 95% |
| #4 | N/A | N/A | <0.16 | 463 | 1169.80 | 95% |
| #5 | 1.6 | 9% | <1.57 | 533 | 1176.90 | 96% |
| #6 | N/A | N/A | <0.16 | 568 | 1166.26 | 95% |
| #7 | 1.6 | 9% | <0.16 | 597 | 1167.90 | 95% |
| #8 | 1.7 | 9% | <0.16 | 612 | 1166.81 | 95% |
| #9 | 1.6 | 9% | <0.16 | 669 | 1167.01 | 95% |
| #10 | 1.6 | 9% | <0.16 | 694 | 1170.77 | 95% |

### Example 12

Filtration of a Crovalimab (Anti-C5 Antibody) Solution with a Silica-Containing Millistak+® HC Pro Synthetic Depth Filter X0SP (Reference Example)

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Millistak+® HC Pro Synthetic Depth Filter X0SP with 23 cm² filter area
3) equilibration buffer: 150 mM acetic acid/Tris, adjusted to pH 5.5 in purified water type II (Advantec CCS-020-D1DS).

Filter Conditioning:

Following steps were performed before the first sample application:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

Experimental Setup:

Following steps were performed sequentially:
1) An intermediate “Affinity Pool pH 5.5” containing crovalimab was applied to the conditioned 23 cm² X0SP filter unit. The mass throughput was 660 g/m². The corresponding calculated volume throughput was 49.6 L/m². The feed flow was adjusted to 200 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flowthrough of the filter was collected for 70 L/m². When the flowthrough of 70 L/m² was reached, the collecting was stopped. Thereby a final filtration pool (comprising the filter flowthrough and the flushing flowthrough) was obtained.
5) Steps 1) to 4) were repeated for further five product filtration cycles without the application of a solution between the filtration cycles. In total, 3.96 kg/m² (6×0.66 kg/m²) antibody were applied to the filter.
6) The procedure has resulted in six fractions. For further analysis the respective fractions were pooled in a final volume of 12 mL (e.g. 4 mL fraction 1, 4 mL fraction 2 and 4 mL fraction 3 resulted in a fraction “Pool #3”).
7) The final pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-12:

| | Final | | | | | |
|---|---|---|---|---|---|---|
| | Filtration | | Photo- | SEC | SEC | SEC |
| | Pool Vol. | Yield | Conc. | [HMW | [MainPeak | [LMW |
| Filtration | [mL] (theo.) | [%] | [g/L] | Area-%] | Area-%] | Area-%] |
| Load X0SP | not | not | 13.37 | 6.62 | 92.22 | 1.17 |
| | applicable | applicable | | | | |
| Fraction #1 | 270.30 | 93.15 | 5.08 | 1.67 | 97.42 | 0.91 |
| w/o Reg. | | | | | | |
| Pool #2 | 270.30 | 95.16 | 5.19 | 2.13 | 96.90 | 0.96 |
| Pool #3 | 270.30 | 97.73 | 5.33 | 2.6 | 96.40 | 0.99 |
| Pool #4 | 270.30 | 96.81 | 5.28 | 2.9 | 96.02 | 0.10 |
| Pool #5 | 270.30 | 97.91 | 5.34 | 3.33 | 95.64 | 1.02 |
| Pool #6 | 270.30 | 98.46 | 5.37 | 3.15 | 95.89 | 0.95 |
| | LEAP | | | | | |
| | Average | | | | | |
| | Converted | LEAP | | | Mass | Yield |
| | Rate [μM | [hydrolytic | DNA | cobas_HCP | Mainpeak | Mainpeak |
| Filtration | MU/h] | activity %] | [ppb] | [ppm] | [mg] | [%] (*) |
| Load X0SP | 18.1 | 100.00 | 5703.59 | 8432.00 | 1359.98 | 100% |
| Fraction #1 | 1.4 | 7.73 | N/A | N/A | 1337.70 | 98.4% |
| w/o Reg. | | | | | | |
| Pool #2 | N/A | N/A | 147.98 | 428.00 | 1359.37 | 100.0% |
| Pool #3 | 3.3 | 18.23 | 457.79 | 834.00 | 1388.83 | 102.1% |
| Pool #4 | 4.6 | 25.41 | 799.24 | 1218.00 | 1370.38 | 100.8% |
| Pool #5 | 5.3 | 29.28 | 1063.67 | 1545.00 | 1380.47 | 101.5% |
| Pool #6 | 6.3 | 34.81 | 1299.81 | 1665.00 | 1391.85 | 102.3% |

N/A = not analyzed
(*) caluclation based on the theoretical volume and not the actual sample volume

### Example 13

Filtration of a Crovalimab (Anti-C5 Antibody) Solution with a Silica-Containing Millistak+® HC Pro Synthetic Depth Filter X0SP, Regenerated with an Acidic Treatment.

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Millistak+® HC Pro Synthetic Depth Filter X0SP, with 23 cm² filter area.
3) equilibration buffer: 150 mM acetic acid/Tris, adjusted to pH 5.5 in purified water type II (Advantec CCS-020-D1DS).
4) acidic regeneration solution: 167 mM acetic acid, 300 mM phosphoric acid, pH 1.34

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing crovalimab was applied to the conditioned 23 cm² X0SP filter unit. The mass throughput was 660 g/m². The corresponding calculated volume throughput was 49.6 L/m². The feed flow was adjusted to 200 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected for 70 L/m². When the flushing flowthrough of 70 L/m² was reached, the collecting was stopped. Thereby a final filtration pool (comprising the filter flowthrough and the flushing flowthrough) was obtained.
5) Thereafter, the acidic regeneration solution was applied to the filter with the same flow rate as the intermediated “Affinity Pool pH 5.5” (7.67 mL/min (200 LMH)). Thus, the contact time was approx. 30 minutes.
6) To remove the regeneration solution from the filter, a water wash with same conditions as described in “filter conditioning: 1)” was carried out. In total, the filter was at a pH value below 2 for approximately 50 minutes.
7) To equilibrate the filter for the next filtration cycle, equilibration buffer was applied with same conditions as described in “filter conditioning: 2)”.
8) Steps 1) to 7) were repeated for further five product filtration cycles (steps 1) to 4)) and further four regeneration cycles (steps 5) to 7)). In total, 3.96 kg/m² (6×0.66 kg/m²) antibody were applied to the filter.
9) The procedure has resulted in six fractions. For further analysis the fractions were pooled in a final volume of 12 mL (e.g. 4 mL fraction 1, 4 mL fraction 2 and 4 mL fraction 3 resulted in a fraction “Pool #3”).
10) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-13:

| | Final | | Photo- | SEC | SEC | SEC |
|---|---|---|---|---|---|---|
| | Filtrat | Yield | Conc. | [HMW | [MainPeak | [LMW |
| Filtration | Vol. [mL] | [%] | [g/L] | Area-%] | Area-%] | Area-%] |
| Load X0SP | not | not | 13.17 | 7.04 | 91.89 | 1.06 |
| | applicable | applicable | | | | |
| #1 w/o Reg. | 291.2 | 98.29 | 5.83 | 1.76 | 97.38 | 0.86 |
| Pool #2 | 291.2 | 98.59 | 5.85 | 1.74 | 97.38 | 0.87 |
| Pool #3 | 291.2 | 99.09 | 5.88 | 1.9 | 97.22 | 0.89 |
| Pool #4 | 291.2 | 99.26 | 5.89 | 1.89 | 97.19 | 0.92 |
| Pool #5 | 291.2 | 99.26 | 5.89 | 1.87 | 97.24 | 0.9 |
| Pool #6 | 291.2 | 100.27 | 5.95 | 1.89 | 97.21 | 0.9 |

| | LEAP | | | |
|---|---|---|---|---|
| | Average | | | |
| | Converted | LEAP | | |
| | Rate [μM | [hydrolytic | | cobas_HCP |
| Filtration | MU/h] | activity %] | DNA [ppb] | [ppm] |
| Load X0SP | 18.1 | 100.00 | 6124.62 | 8804.00 |
| #1 w/o | N/A | N/A | <0.14 | 18.00 |
| Pool #2 | N/A | N/A | <0.14 | 39.00 |
| Pool #3 | 1.3 | 7.18 | <0.14 | 50.00 |
| Pool #4 | N/A | N/A | <0.15 | 68.00 |
| Pool #5 | N/A | N/A | <0.15 | 80.00 |
| Pool #6 | 1.5 | 8.29 | <0.15 | 80.00 |

| | Mass | Yield |
|---|---|---|
| | Mainpeak | Mainpeak |
| Filtration | [mg] | [%] (*) |
| Load X0SP | 1587.77 | 100% |
| #1 w/o Reg. | 1653.22 | 104% |
| Pool #2 | 1658.89 | 104% |
| Pool #3 | 1656.16 | 104% |
| Pool #4 | 1511.31 | 95% |
| Pool #5 | 1509.26 | 95% |
| Pool #6 | 1542.76 | 97% |

(*) caluclation based on the theoretical volume and not the actual sample volume

### Example 14

Filtration of a Crovalimab (Anti-C5 Antibody) Solution with a Silica-Containing Millistak+® HC Pro Synthetic Depth Filter X0SP, with Water and Buffer Regeneration (without Alkaline or Acidic Filter Regeneration), with a Pre-Incubation with 1 M NaOH for Four Hours Prior to First Use

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Millistak+® HC Pro Synthetic Depth Filter X0SP with 23 cm² filter area.
3) equilibration buffer: 150 mM acetic acid/tris, adjusted to pH 5.5 in purified water type II (Advantec CCS-020-D1DS).
4) regeneration solution: water and equilibration buffer

Filter Derivatization:

Following steps were performed sequentially:
1) The X0SP filter was flown through with 1 M NaOH, deaerated and the flow was stopped until a final incubation time with 1 M NaOH of 4 hours was reached. The flow was 10.0 mL/min at a max. feed pressure of 5.0 bar.
2) 100 L/m² water were applied to/flown through the filter. The washing flow was 10.0 mL/min at a max. feed pressure of 5.0 bar.
3) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing crovalimab was applied to the derivatized 23 cm² X0SP filter unit. The mass throughput was 660 g/m². The corresponding calculated volume throughput was 50.65 L/m². The feed flow was adjusted to 200 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected for 70 L/m². When the flushing flowthrough of 70 L/m² was reached, the collecting was stopped. Thereby a final filtration pool (comprising the filter flowthrough and the flushing flowthrough) was obtained.
5) To prepare the filter for the next filtration cycle, water and equilibration buffer were applied with same conditions as described in “filter derivatization: 2) and 3)”.
6) Steps 1) to 4) were repeated for further five product filtration cycles and step 5) for further four regeneration cycles. In total, 3.96 kg/m² (6×0.66 kg/m²) antibody were applied to the filter.
7) The procedure has resulted in six fractions. For further analysis the fractions were pooled in a final volume of 12 mL (e.g. 4 mL fraction 1, 4 mL fraction 2 and 4 mL fraction 3 resulted in a fraction “Pool #3”).
8) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-14:

| | Final | | | | | |
|---|---|---|---|---|---|---|
| | Filtration | | Photo- | SEC | SEC | SEC |
| | Pool Vol. | Yield | Conc. | [HMW | [MainPeak | [LMW |
| Filtration | [mL] | [%] | [g/L] | Area-%] | Area-%] | Area-%] |
| Load X0SP | not | not | 13.03 | 5.13 | 94.05 | 0.82 |
| | applicable | applicable | | | | |
| Fraction #1 | 250.20 | 86.86 | 5.27 | 1.69 | 97.5 | 0.81 |
| Pool #2 | 256.80 | 90.51 | 5.42 | 1.82 | 97.44 | 0.74 |
| Pool #3 | 257.50 | 91.16 | 5.43 | 2.4 | 96.67 | 0.92 |
| Pool #4 | 257.70 | 91.17 | 5.45 | 1.86 | 97.42 | 0.73 |
| Pool #5 | 257.80 | 92.59 | 5.49 | 1.88 | 97.35 | 0.76 |
| Pool #6 | 258.50 | 93.07 | 5.51 | 1.3 | 97.97 | 0.72 |
| Pool #7 | 258.90 | 93.71 | 5.54 | 2.06 | 97.22 | 0.72 |
| Pool #8 | 259.30 | 93.66 | 5.53 | 1.99 | 97.28 | 0.73 |
| Pool #9 | 259.40 | 93.92 | 5.54 | 2.29 | 97.00 | 0.72 |
| | LEAP | | | | | |
| | Average | | | | | |
| | Converted | LEAP | | | Mass | Yield |
| | Rate | [hydrolytic | | cobas_HCP | Mainpeak | Mainpeak |
| Filtration | [μM MU/h] | activity %] | DNA [ppb] | [ppm] | [mg] | [%] |
| Load X0SP | 15.2 | 100 | 4328.47 | 9015.00 | 1427.67 | 100.0% |
| Fraction #1 | 2.7 | 17.76 | <0.15 | 684.00 | 1285.59 | 90.0% |
| Pool #2 | 3.1 | 20.39 | <0.15 | 858.00 | 1356.22 | 95.0% |
| Pool #3 | 3.2 | 21.05 | <0.15 | 1145.00 | 1351.66 | 94.7% |
| Pool #4 | 3.7 | 24.34 | 11.27 | 1184.00 | 1368.23 | 95.8% |
| Pool #5 | 3.74 | 24.61 | 76.5 | 1428.00 | 1377.82 | 96.5% |
| Pool #6 | 4.2 | 27.63 | 195.64 | 1904.00 | 1395.42 | 97.7% |
| Pool #7 | 5.4 | 35.53 | 351.99 | 3131.00 | 1394.43 | 97.7% |
| Pool #8 | 6 | 39.47 | 470.16 | 2809.00 | 1394.93 | 97.7% |
| Pool #9 | 6.2 | 40.79 | 667.87 | 3185.00 | 1393.96 | 97.6% |

### Example 15

Filtration of a Crovalimab (Anti-C5 Antibody) Solution with a Silica-Containing Pall PDD1 SUPRAcap 50 Filter (Reference Example)

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Pall PDD1 SUPRAcap 50 (SC050PDD1) filter with 22 cm² filter area.
3) equilibration buffer: 150 mM acetic acid/Tris, adjusted to pH 5.5 in purified water type II (Advantec CCS-020-D1DS)

Filter Conditioning:

Following steps were performed before the first sample application:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing crovalimab was applied to the conditioned 22 cm² PDD1 filter unit. The mass throughput was 690 g/m². The corresponding calculated volume throughput was 51.9 L/m². The feed flow was adjusted to 191 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flowthrough of the filter was collected for 70 L/m². When the flowthrough of 70 L/m² was reached, the collecting was stopped. Thereby a final filtration pool (comprising the filter flowthrough and the flushing flowthrough) was obtained.
5) Steps 1) to 4) were repeated for further five product filtration cycles without the application of a solution between the filtration cycles. In total, 4.14 kg/m² (6×0.69 kg/m²) antibody were applied to the filter.
6) The procedure has resulted in six fractions. For further analysis the fractions were pooled in a final volume of 12 mL (e.g. 4 mL fraction 1, 4 mL fraction 2 and 4 mL fraction 3 resulted in a fraction “Pool #3”).
7) The final pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-15:

| | Final | | | | | |
|---|---|---|---|---|---|---|
| | Filtration | | | | | |
| | Pool Vol. | | Photo- | SEC | SEC | SEC |
| | [mL] | Yield | Conc. | [HMW | [MainPeak | [LMW |
| Filtration | (theo.) | [%] | [g/L] | Area-%] | Area-%] | Area-%] |
| Load PDD1 | not | not | 13.29 | 6.23 | 92.56 | 1.12 |
| | applicable | applicable | | | | |
| Fraction #1 | 274.2 | 97.92 | 5.42 | 1.28 | 97.71 | 1.00 |
| Pool #2 | 274.2 | 99.37 | 5.5 | 1.67 | 97.38 | 0.95 |
| Pool #3 | 274.2 | 101.53 | 5.62 | 2.58 | 96.34 | 1.08 |
| Pool #4 | 274.2 | 100.99 | 5.59 | 3.05 | 95.98 | 0.97 |
| Pool #5 | 274.2 | 104.06 | 5.76 | 3.52 | 95.38 | 1.11 |
| Pool #6 | 274.2 | 102.44 | 5.67 | 3.76 | 95.15 | 1.10 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] (*) |
| Load PDD1 | 18.1 | 100 | 5658.39 | 8817 | 1404.80 | 100% |
| Fraction #1 | 1.1 | 6.08 | <1.48 | 52 | 1452.13 | 103% |
| Pool #2 | 1.7 | 9.39 | 65.45 | 241 | 1468.59 | 105% |
| Pool #3 | 3.3 | 18.23 | 537.37 | 1033 | 1484.60 | 106% |
| Pool #4 | 4.4 | 24.31 | 991.06 | 1436 | 1471.16 | 105% |
| Pool #5 | 4.6 | 25.41 | 1427.08 | 1653 | 1506.42 | 107% |
| Pool #6 | 5.8 | 32.04 | 1696.65 | 1782 | 1479.31 | 105% |

(*) caluclation based on the theoretical volume and not the actual sample volume

### Example 16

Filtration of a Crovalimab (Anti-C5 Antibody) Solution with a Silica-Containing Pall PDD1 SUPRAcap 50 Filter, Regenerated with an Acidic Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Pall PDD1 SUPRAcap 50 (SC050PDD1) filter with 22 cm² filter area.
3) equilibration buffer: 150 mM acetic acid/Tris, adjusted to pH 5.5 in purified water type II (Advantec CCS-020-D1DS).
4) acidic regeneration solution: 167 mM acetic acid, 300 mM phosphoric acid, pH 1.34

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing crovalimab was applied to the conditioned 22 cm² PDD1 filter unit. The mass throughput was 690 g/m². The corresponding calculated volume throughput was 52.39 L/m². The feed flow was adjusted to 191 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected for 70 L/m². When the flushing flowthrough of 70 L/m² was reached, the collecting was stopped. Thereby a final filtration pool (comprising the filter flowthrough and the flushing flowthrough) was obtained.
5) Thereafter, the acidic regeneration solution was applied to the filter with the same flow rate as the intermediated “Affinity Pool pH 5.5” (7.67 mL/min (191 LMH)). Thus, the contact time was approx. 30 minutes.
6) To remove the regeneration solution from the filter, a water wash with same conditions as described in “filter conditioning: 1)” was carried out. In total, the filter was at a pH value below 2 for approximately 50 minutes.
7) To equilibrate the filter for the next filtration cycle, equilibration buffer was applied with same conditions as described in “filter conditioning: 2)”.
8) Steps 1) to 7) were repeated for further five product filtration cycles (steps 1) to 4)) and further four regeneration cycles (steps 5) to 7)). In total, 4.14 kg/m² (6×0.69 kg/m²) antibody were applied to the filter.
9) The procedure has resulted in six fractions. For further analysis the fractions were pooled in a final volume of 12 mL (e.g. 4 mL fraction 1, 4 mL fraction 2 and 4 mL fraction 3 resulted in a fraction “Pool #3”).
10) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-16:

| | Final | | Photo- | SEC | SEC | SEC |
|---|---|---|---|---|---|---|
| | Filtrat | Yield | Conc. | [HMW | [MainPeak | [LMW |
| Filtration | Vol. [mL] | [%] | [g/L] | Area-%] | Area-%] | Area-%] |
| Load PDD1 | not | not | 13.17 | 6.56 | 92.38 | 1.07 |
| | applicable | applicable | | | | |
| Fraction #1 | 275.26 | 99.01 | 5.46 | 1.29 | 97.77 | 0.94 |
| w/o Reg. | | | | | | |
| Pool #2 | 275.26 | 101.73 | 5.61 | 1.28 | 97.85 | 0.86 |
| Pool #3 | 275.26 | 100.64 | 5.55 | 1.60 | 97.39 | 1.00 |
| Pool #4 | 275.26 | 100.82 | 5.56 | 1.66 | 97.32 | 1.01 |
| Pool #5 | 275.26 | 101.55 | 5.6 | 1.71 | 97.27 | 1.03 |
| Pool #6 | 275.26 | 101.00 | 5.57 | 1.75 | 97.24 | 1.01 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] |
| Load PDD1 | 18.1 | 100 | 6590.74 | 8277 | 1402.30 | 100% |
| Fraction #1 | 1.1 | 6.08 | <1.47 | 52 | 1469.40 | 105% |
| w/o Reg. | | | | | | |
| Pool #2 | N/A | N/A | <1.43 | 176 | 1511.01 | 108% |
| Pool #3 | 1.3 | 7.18 | <1.44 | 178 | 1487.82 | 106% |
| Pool #4 | N/A | N/A | <1.44 | 281 | 1489.43 | 106% |
| Pool #5 | N/A | N/A | <1.43 | 313 | 1499.37 | 107% |
| Pool #6 | 1.3 | 7.18 | <1.44 | 354 | 1490.88 | 106% |

N/A = not analyzed

### Example 17

Filtration of a Crovalimab (Anti-C5 Antibody) Solution with a Silica-Containing Pall PDD1 SUPRAcap 50 Filter, Regenerated with an Acidic Treatment, without Intermediate Water Flush for Reducing Process Time

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Pall PDD1 SUPRAcap 50 (SC050PDD1) filter with 22 cm² filter area; Lot.: 104072586.
3) equilibration buffer: 150 mM acetic acid/Tris, adjusted to pH 5.5 in purified water type II (Advantec CCS-020-D1DS).
4) acidic regeneration solution: 167 mM acetic acid, 300 mM phosphoric acid, pH 1.5

Filter Conditioning:

Following step was performed:
1) 200 L/m² equilibration buffer were applied to/flown through the filter (equilibration of the system and the filter). The equilibration flow was 10.0 mL/min at a max. feed pressure of 5.0 bar.

Experimental Setup:

Following steps were performed sequentially:
1) An intermediate “Affinity Pool pH 5.5” containing crovalimab was applied to the conditioned 22 cm² PDD1 filter unit. The mass throughput was 627 g/m². The corresponding calculated volume throughput was 48 L/m². The feed flow was adjusted to 191 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected for 70 L/m². When the flushing flowthrough of 70 L/m² was reached, the collecting was stopped. Thereby a final filtration pool (comprising the filter flowthrough and the flushing flowthrough) was obtained.
5) Thereafter, the acidic regeneration solution was applied to the filter with the same flow rate as the intermediated “Affinity Pool pH 5.5” (7.67 mL/min (191 LMH)). Thus, the contact time was approx. 30 minutes.
6) To equilibrate the filter for the next filtration cycle, equilibration buffer was applied with same conditions as described in “filter conditioning: 1)” was carried out. In total, the filter was at a pH value below 2 for approximately 50 minutes.
7) Steps 1) to 6) were repeated for further five product filtration cycles (steps 1) to 4)) and further four regeneration cycles (steps 5) and 6)). In total, 3.762 kg/m² (6×0.627 kg/m²) antibody were applied to the filter.
8) The procedure has resulted in six fractions. For further analysis the fractions were pooled in a final volume of 12 mL (e.g. 4 mL fraction 1, 4 mL fraction 2 and 4 mL fraction 3 resulted in a fraction “Pool #3”).
9) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-17:

| | Final | | Photo- | SEC | SEC | SEC |
|---|---|---|---|---|---|---|
| | Filtrat | Yield | Conc. | [HMW | [MainPeak | [LMW |
| Filtration | Vol. [mL] | [%] | [g/L] | Area-%] | Area-%] | Area-%] |
| Load PDD1 | not | not | 13.07 | 5.19 | 93.98 | 0.84 |
| | applicable | applicable | | | | |
| Fraction #1 | 245.2 | 90.25 | 5.08 | 1.26 | 98.00 | 0.73 |
| w/o Reg. | | | | | | |
| Pool #2 | 248.6 | 92.13 | 5.15 | 1.47 | 97.65 | 0.88 |
| Pool #3 | 249.0 | 92.39 | 5.15 | 1.55 | 97.64 | 0.80 |
| Pool #4 | 249.5 | 92.39 | 5.14 | 1.39 | 97.85 | 0.76 |
| Pool #5 | 249.5 | 92.85 | 5.16 | 1.39 | 97.83 | 0.78 |
| Pool #6 | 249.9 | 93.13 | 5.17 | 1.41 | 97.83 | 0.76 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] |
| Load PDD1 | 13.5 | 100 | 5524.1 | 9359 | 1297.10 | 100% |
| Fraction #1 | 1 | 7.41 | <0.16 | 35 | 1220.70 | 94% |
| w/o Reg. | | | | | | |
| Pool #2 | 1.2 | 8.89 | <0.16 | 95 | 1250.20 | 96% |
| Pool #3 | 1.1 | 8.15 | <1.55 | 180 | 1252.09 | 97% |
| Pool #4 | 1.4 | 10.37 | <1.56 | 222 | 1254.86 | 97% |
| Pool #5 | 1.3 | 9.63 | <1.55 | 253 | 1259.48 | 97% |
| Pool #6 | 1.2 | 8.89 | <1.55 | 359 | 1263.95 | 97% |

### Example 18

Filtration of a Crovalimab (Anti-C5 Antibody) Solution with a Zeta Plus™ Biocap VR02 without Intermediate Flush (Reference Example)

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Zeta Plus™ Biocap VR02 filter with 25 cm² filter area; (Lot.: 3923451).
3) equilibration buffer: 150 mM acetic acid/Tris, adjusted to pH 5.5 in purified water type II (Advantec CCS-020-D1DS).

Filter Conditioning:

Following steps were performed before the first sample application:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing crovalimab was applied to the conditioned 25 cm² VR02 filter unit. The mass throughput was 659 g/m². The corresponding calculated volume throughput was 54.8 L/m². The feed flow was adjusted to 184 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flowthrough of the filter was collected for 70 L/m². When the flowthrough of 70 L/m² was reached, the collecting was stopped. Thereby a final filtration pool (comprising the filter flowthrough and the flushing flowthrough) was obtained.
5) Steps 1) to 4) were repeated for further five product filtration cycles without the application of a solution between the filtration cycles. In total, 3.95 kg/m² (6×0.659 kg/m²) antibody were applied to the filter.
6) The procedure has resulted in six fractions. For further analysis the fractions were pooled in a final volume of 12 mL (e.g. 4 mL fraction 1, 4 mL fraction 2 and 4 mL fraction 3 resulted in a fraction “Pool #3”).
7) The final pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA SE-HPLC (size exclusion HPLC)

The results are shown in the following Table X-18:

| | Final | | | | | |
|---|---|---|---|---|---|---|
| | Filtration | | Photo- | SEC | SEC | SEC |
| | Pool Vol. | Yield | Conc. | [HMW | [MainPeak | [LMW |
| Filtration | [mL] (theo.) | [%] | [g/L] | Area-%] | Area-%] | Area-%] |
| Load VR02 | not | not | 13.07 | 4.64 | 94.54 | 0.83 |
| | applicable | applicable | | | | |
| Fraction #1 | 274.9 | 95.63 | 5.74 | 1.97 | 97.23 | 0.8 |
| Pool #2 | 277.0 | 97.84 | 5.85 | 3.71 | 95.33 | 0.95 |
| Pool #3 | 277.3 | 100.18 | 5.98 | 4.1 | 94.94 | 0.96 |
| Pool #4 | 277.5 | 98.77 | 5.89 | 4.36 | 94.66 | 0.98 |
| Pool #5 | 277.6 | 99.33 | 5.92 | 4.51 | 94.49 | 1.00 |
| Pool #6 | 277.5 | 99.21 | 5.91 | 4.67 | 94.37 | 0.97 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] |
| Load VR02 | 15.5 | 100% | 4697.78 | 9430.00 | 1557.40 | 100% |
| Fraction #1 | 4.5 | 29% | 616.72 | 2253.00 | 1534.22 | 99% |
| Pool #2 | 8.4 | 54% | 2037.61 | 3836.00 | 1544.77 | 99% |
| Pool #3 | 10.6 | 68% | 2729.1 | 4244.00 | 1574.35 | 101% |
| Pool #4 | 11.2 | 72% | 2964.35 | 4929.00 | 1547.19 | 99% |
| Pool #5 | 12.9 | 83% | 3378.38 | 5013.00 | 1552.84 | 100% |
| Pool #6 | 13.1 | 85% | 3722.5 | 5170.00 | 1547.69 | 99% |

### Example 19

Filtration of a Crovalimab (Anti-C5 Antibody) Solution with a Zeta Plus™ Biocap VR02 Filter, with Water and Buffer Application (without Alkaline or Acidic Filter Regeneration)—Comparative Example

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Zeta Plus™ Biocap VR02 filter with 25 cm² filter area; (Lot.: 3923451).
3) Equilibration buffer: 150 mM acetic acid/Tris, adjusted to pH 5.5 in purified water type II (Advantec CCS-020-D1DS).
4) intermediate solution: water and equilibration buffer

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

### Experimental Setup

Following steps were performed sequentially:
1) An intermediate “Affinity Pool pH 5.5” containing crovalimab was applied to the equilibrated 25 cm² VR02 filter unit. The mass throughput was 659 g/m². The corresponding calculated volume throughput was 54.8 L/m². The feed flow was adjusted to 184 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected for 70 L/m². When the flushing flowthrough of 70 L/m² was reached, the collecting was stopped. Thereby a final filtration pool (comprising the filter flowthrough and the flushing flowthrough) was obtained.
5) To prepare the filter for the next filtration cycle, water and equilibration buffer were applied with same conditions as described in “filter conditioning: 1) and 2)”.
6) Steps 1) to 4) were repeated for further five product filtration cycles and step 5) for further four regeneration cycles. In total, 3.95 kg/m² (6×0.659 kg/m²) antibody were applied to the filter.
7) The procedure has resulted in six fractions. For further analysis the fractions were pooled in a final volume of 12 mL (e.g. 4 mL fraction 1, 4 mL fraction 2 and 4 mL fraction 3 resulted in a fraction “Pool #3”).
8) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-19:

| | Final | | | | | |
|---|---|---|---|---|---|---|
| | Filtrat | | Photo- | SEC | SEC | SEC |
| | Vol. | Yield | Conc. | [HMW | [MainPeak | [LMW |
| Filtration | [mL] | [%] | [g/L] | Area-%] | Area-%] | Area-%] |
| Load VR02 | not | not | 13.07 | 5.5 | 93.56 | 0.95 |
| | applicable | applicable | | | | |
| Fraction #1 | 275.8 | 97.79 | 5.85 | 2.09 | 97.17 | 0.74 |
| Pool #2 | 277.4 | 100.08 | 5.97 | 3.89 | 95.14 | 0.96 |
| Pool #3 | 277.4 | 100.36 | 5.98 | 4.27 | 94.75 | 0.98 |
| Pool #4 | 277.3 | 97.87 | 5.83 | 4.5 | 94.53 | 0.97 |
| Pool #5 | 277.4 | 99.41 | 5.92 | 4.63 | 94.41 | 0.97 |
| Pool #6 | 277.6 | 98.6 | 5.87 | 4.76 | 94.27 | 0.98 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] |
| Load VR02 | 15.5 | 100% | 4483.55 | 9715.00 | 1541.25 | 100% |
| Fraction #1 | 4.7 | 30% | 762.39 | 2510.00 | 1567.77 | 102% |
| Pool #2 | 7.0 | 45% | 1805.7 | 3923.00 | 1575.59 | 102% |
| Pool #3 | 10.2 | 66% | 2588.63 | 4259.00 | 1571.76 | 102% |
| Pool #4 | 11.5 | 74% | 3138.94 | 4675.00 | 1528.23 | 99% |
| Pool #5 | 11.4 | 74% | 2949.32 | 4930.00 | 1550.41 | 101% |
| Pool #6 | 12.3 | 79% | 8926.75 | 5265.00 | 1536.14 | 100% |

### Example 20

Filtration of a Crovalimab (Anti-C5 Antibody) Solution with a Zeta Plus™ Biocap VR02 Filter, Regenerated with an Acidic Treatment

Materials:
1) Experiments were performed with an Äkta Avant 150 (Cytiva, Uppsala, Sweden) chromatographic skid. The filter was installed to the column valve instead of a column. Pressure, pH value, conductivity, OD280 were monitored. The applied volume was regulated by a sample pump.
2) Zeta Plus™ Biocap VR02 filter with 25 cm² filter area; Lot.: 3923451.
3) Equilibration buffer: 150 mM acetic acid/Tris, adjusted to pH 5.5 in purified water type II (Advantec CCS-020-D1DS).
4) Acidic regeneration solution: 167 mM acetic acid, 300 mM phosphoric acid, pH 1.34

Filter Conditioning:

Following steps were performed sequentially:
1) 100 L/m² water were applied to/flown through the filter. Thereafter the filter was deaerated. The washing flow was 10.0 mL/min at a max. feed pressure of 5.0 bar.
2) 100 L/m² equilibration buffer were applied to/flown through the filter (equilibration of the system and the filter). The equilibration flow was 10.0 mL/min at a max. feed pressure of 5.0 bar.

Experimental Setup:

Following steps were performed sequentially:
1) An intermediate “Affinity Pool pH 5.5” containing crovalimab was applied to the conditioned 25 cm² VR02 filter unit. The mass throughput was 659 g/m². The corresponding calculated volume throughput was 54.8 L/m². The feed flow was adjusted to 184 LMH (liter per square meter per hour; L*m⁻²*h⁻¹). This resulted in a calculated feed flow of 7.67 mL/min. The maximum feed pressure was 5.0 bar.
2) The filter flowthrough was not collected until the OD280 (280 nm) exceeded 0.5 AU determined using a 1 cm light path length UV-cell. After exceeding the threshold signal level, the flowthrough was collected in a tared and sterile bottle (Nalgene) resulting in a filtration pool.
3) When the intended loading volume had been applied, the filter was flushed with equilibration buffer (washing out of remaining protein solution).
4) The flushing flowthrough was collected for 70 L/m². When the flushing flowthrough of 70 L/m² was reached, the collecting was stopped. Thereby a final filtration pool (comprising the filter flowthrough and the flushing flowthrough) was obtained.
5) Thereafter, the acidic regeneration solution was applied to the filter with the same flow rate as the intermediated “Affinity Pool pH 5.5” (7.67 mL/min (184 LMH)). Thus, the contact time was approx. 30 minutes.
6) To remove the regeneration solution from the filter, a water wash with same conditions as described in “filter conditioning: 1)” was carried out. In total, the filter was at a pH value below 2 for approximately 50 minutes.
7) To equilibrate the filter for the next filtration cycle, equilibration buffer was applied with same conditions as described in “filter conditioning: 2)”.
8) Steps 1) to 7) were repeated for further five product filtration cycles (steps 1) to 4)) and further four regeneration cycles (steps 5) to 7)). In total, 3.95 kg/m² (6×0.659 kg/m²) antibody were applied to the filter.
9) The procedure has resulted in six fractions. For further analysis the fractions were pooled in a final volume of 12 mL (e.g. 4 mL fraction 1, 4 mL fraction 2 and 4 mL fraction 3 is “Pool #3”).
10) The separate final filtration pools were kept for analysis at −80° C.

Analyses and Results:

The following analyses were performed with the respective final filtration pools:
protein concentration (using the absorbance of 1.44 at 1 mg/ml as reference)
total product yield, calculated out of loaded mass, final filtration pool volume and final filtration pool protein concentration
LEAP (Lipase Activity Assay; hydrolytic activity)
CHOP values (host cell proteins) by cobas_HCP
DNA values (host cell DNA) by qPRC_DNA
SE-HPLC (size exclusion HPLC)

The results are shown in the following Table X-20:

| | Final | | | | | |
|---|---|---|---|---|---|---|
| | Filtrat | | Photo- | SEC | SEC | SEC |
| | Vol. | Yield | Conc. | [HMW | [MainPeak | [LMW |
| Filtration | [mL] | [%] | [g/L] | Area-%] | Area-%] | Area-%] |
| Load VR02 | not | not | 13.07 | 5.64 | 93.42 | 0.94 |
| | applicable | applicable | | | | |
| Fraction #1 | 272.8 | 95.90 | 5.8 | 2.08 | 97.15 | 0.77 |
| w/o Reg. | | | | | | |
| Pool #2 | 274.0 | 96.44 | 5.82 | 2.11 | 97.12 | 0.76 |
| Pool #3 | 274.4 | 97.22 | 5.86 | 2.15 | 97.05 | 0.8 |
| Pool #4 | 274.8 | 98.64 | 5.94 | 2.19 | 97.01 | 0.79 |
| Pool #5 | 275.1 | 97.23 | 5.85 | 2.23 | 97.02 | 0.75 |
| Pool #6 | 275.5 | 99.13 | 5.96 | 3.24 | 95.81 | 0.94 |
| | LEAP | | | | | |
| | Average | | | | | |
| | Converted | LEAP | | | Mass | Yield |
| | Rate [μM | [hydrolytic | | cobas_HCP | Mainpeak | Mainpeak |
| Filtration | MU/h] | activity %] | DNA [ppb] | [ppm] | [mg] | [%] |
| Load VR02 | 15.5 | 100% | 1371.08 | 8962.00 | 1538.95 | 100% |
| Fraction #1 | 4.3 | 28% | 817.24 | 2233.00 | 1537.15 | 100% |
| w/o Reg. | | | | | | |
| Pool #2 | 4.0 | 26% | 934.71 | 2396.00 | 1548.75 | 101% |
| Pool #3 | 4.5 | 29% | 1020.48 | 2436.00 | 1560.55 | 101% |
| Pool #4 | 5.3 | 34% | 1033.67 | 2490.00 | 1583.51 | 103% |
| Pool #5 | 5.2 | 34% | 1001.71 | 2782.00 | 1561.38 | 101% |
| Pool #6 | 5.1 | 33% | 946.66 | 2677.00 | 1573.18 | 102% |

### Example 21

Hydrolytic Activity Determination—Lipase Activity Assay (LEAP):

The lipase activity was determined by monitoring the conversion of a substrate, such as a nonfluorescent substrate, to a detectable product of the hydrolytic enzyme, such as a fluorescent product.

In more detail, with the LEAP assay hydrolase activity in samples was determined. This was done by monitoring the conversion of a fluorogenic substrate ‘4-Methylumbelliferyl Caprylate’ (4-MU-C8, available from Chem Impex Int'l Inc Art. Nr. 01552) by cleavage of the ester bond by hydrolases present in the sample into a fluorescent moiety, i.e. 4-Methylumbelliferyl (4-MU). Cleaved 4-MU-C8, i.e. 4-MU, was excited with a light of wavelength 355 nm. The emitted radiation at a different wavelength of 460 nm was recorded on Tecan Infinite® 200 PRO device as readout. The determination was performed at 37° C. for 2 hours with recording every 10 mins to calculate the rate of substrate hydrolysis.

The sample to be analyzed was at first buffer exchanged to 150 mM Tris-Cl, pH 8.0, by using Amicon Ultra-0.5 ml centrifugal filter units (10,000 Da cut-off, Merck Millipore, Art. Nr. UFC501096). The assay reaction mixture constituted of 80 μL reaction buffer (150 mM Tris-Cl, pH 8.0, 0.25% (w/v) Triton X-100, 0.125% (w/v) Gum Arabic), 10 μL 4-MU-C8 substrate solution (1 mM in DMSO), and 10 μL protein containing sample. The protein samples' concentration were adjusted to be in the range between 1-30 g/L and 2-3 dilution series were performed for each determination. Each reaction was set up at least in duplicates in 96-well half-area polystyrene plates (black with lid and clear flat bottom, Corning Incorporated Art. Nr. 3882).

1. A method for purifying a therapeutic polypeptide, characterized in that the method comprises the following steps:
a) filtering an aqueous composition containing said therapeutic polypeptide and impurities through a depth filter, recovering the flow-through and thereby obtaining said purified therapeutic polypeptide,
b) contacting said depth filter with an acidic solution and thereby regenerating the depth filter,
and
c) repeating steps a) and b) one or more times.

2. A method for producing a therapeutic polypeptide, characterized in that the method comprises the following steps:
a) filtering an aqueous composition containing said therapeutic polypeptide and impurities through a depth filter, recovering the flow-through and thereby obtaining said therapeutic polypeptide,
b) contacting said depth filter (after step a)) with an acidic solution and thereby regenerating the depth filter,
and
c) repeating steps a) and b) one or more times.

3. The method according to any one of claims 1 to 2, wherein the acidic solution of step b) has a pH value between and including 1 to 3.

4. The method according to claim 1 or 3, wherein the acidic solution of step b) is a solution comprising phosphoric acid.

5. The method according to any one of claims 1 to 4, wherein the acidic solution of step b) is a solution comprising phosphoric acid and acetic acid.

6. The method according to any one of claims 1 to 5, wherein the acidic solution of step b) comprises phosphoric acid in a concentration of about 0.1 M to about 0.8 M, or about 0.2 M to about 0.7 M, or about 0.4 M to 0.6 M.

7. The method according to any one of claims 1 to 6, wherein the acidic solution of step b) comprises acetic acid in a concentration of about 10 mM to 2 M or about 20 mM to 1.5 M or about 50 mM to 1 M, or about 80 mM to 800 mM.

8. The method according to any one of claims 1 to 7, wherein the acidic solution of step b) comprises phosphoric acid in a concentration of about 300 mM and acetic acid in a concentration of about 167 mM.

9. The method according to any of claims 1 to 8, wherein the depth filter comprises silica.

10. The method according to any one of claims 1 to 9, wherein the method reduces the enzymatic hydrolysis activity rate.

11. The method according to any one of claims 1 to 10, wherein the depth filter comprises material that is selected from the group of
(i) polyacrylic fiber and silica;
(ii) cellulose fibers, diatomaceous earth, and perlite, and
(iii) cellulose fiber and charged surface groups.

12. The method according to any one of claims 1 to 11, wherein the depth filter is selected from the group consisting of an X0SP depth filter, or a PDD1 depth filter, or a VR02 depth filter.

13. The method according to any one of claims 1 to 12, wherein the depth filter is contacted with the regeneration solution of step b) for about 20 min or more, 30 min or more, 40 min or more, 50 min or more or 60 min or more.

14. Use of an acidic solution for regeneration of a depth filter that is being used at least two times in the purification of a therapeutic polypeptide.

15. Use of an alkaline solution for regeneration of a depth filter that is being used at least two times in the purification of a therapeutic polypeptide.
